(19) United States
(12) Patent Application Publication
(10) Pub. No.: US 2026/0174776 A1
(43) Pub. Date: Jun. 25, 2026

(54) METHODS OF TREATING LIPEDEMA INCLUDING AKR1C1 AS A THERAPEUTIC TARGET

(71) Applicant: MAGI EUREGIO SCS, Bolzano (IT)

(72) Inventors: Matteo Bertelli, San Felice del Benaco (IT); Elena Manara, Dro (IT); Paolo Enrico Maltese, Rovereto (IT); Sandro Michelini, Roma (IT); Serena Michelini, Roma (IT); Maria Rachele Ceccarini, Marsciano (IT); Pietro Chiurazzi, Roma (IT); Karen Louise Herbst, Tucson, AZ (US)

(73) Assignee: MAGI EUREGIO SCS, Bolzano (IT)

(21) Appl. No.: 19/549,798

(22) Filed: Feb. 25, 2026

Related U.S. Application Data
(62) Division of application No. 17/734,708, filed on May 2, 2022, now abandoned.
(60) Provisional application No. 63/183,313, filed on May 3, 2021.

(51) Int. Cl. A61K 31/551; A61K 31/05; A61K 31/121; A61K 31/122; A61K 31/138; A61K 31/16; A61K 31/167; A61K 31/19; A61K 31/192; A61K 31/196; A61K 31/201; A61K 31/22; A61K 31/231; A61K 31/352; A61K 31/353; A61K 31/365; A61K 31/366; A61K 31/37; A61K 31/40; A61K 31/4035; A61K 31/405; A61K 31/407; A61K 31/415; A61K 31/5517; A61K 31/566; A61K 31/567; A61K 31/57; A61K 31/575; A61K 31/585; A61K 31/60; A61K 31/603; A61K 31/616; A61K 31/7034; A61K 31/704; A61K 31/7048; A61K 33/40; A61K 36/05; A61K 36/38; A61K 36/82; C12Q 1/32; C12Q 1/6869; C12Q 1/6883; G01N 33/68

(52) U.S. Cl. CPC A61K 31/551; A61K 31/05; A61K 31/121; A61K 31/122; A61K 31/138; A61K 31/16; A61K 31/167; A61K 31/19; A61K 31/192; A61K 31/196; A61K 31/201; A61K 31/22; A61K 31/231; A61K 31/352; A61K 31/353; A61K 31/365; A61K 31/366; A61K 31/37; A61K 31/40; A61K 31/4035; A61K 31/405; A61K 31/407; A61K 31/415; A61K 31/5517; A61K 31/566; A61K 31/567; A61K 31/57; A61K 31/575; A61K 31/585; A61K 31/60; A61K 31/603; A61K 31/616; A61K 31/7034; A61K 31/704; A61K 31/7048; A61K 33/40; A61K 36/05; A61K 36/38; A61K 36/82; C12Q 1/32; C12Q 1/6883; G01N 33/6893; C12Q 1/6869; C12Q 2600/156; C12Q 2600/158; G01N 2800/044

(57) ABSTRACT

The present invention identifies AKR1C1 as the first lipedema-associated gene. The invention provides methods for diagnosing or assessing an individual's susceptibility to lipedema by the analysis of the AKR1C1 gene or the expression levels of its product and related metabolites. Also provided are therapeutic methods for treating a patient or methods for prophylactically treating an individual susceptible to lipedema.

### CROSS-REFERENCE TO RELATED APPLICATION

This application is a Divisional of co-pending U.S. application Ser. No. 17/734,708, filed on May 2, 2022, which claims the benefit under 35 U.S.C. § 119(e) to U.S. Provisional Application No. 63/183,313, filed on May 3, 2021. Each of the above applications is hereby expressly incorporated by reference, in its entirety, into the present application.

### REFERENCE TO SEQUENCE LISTING SUBMITTED VIA EFS-WEB

This application includes an electronically submitted sequence listing in .xml format. The .xml file contains a sequence listing entitled “6847-0107PUS4_Sequence_Listing.xml” created on Feb. 19, 2026 and is 53,381 bytes in size. The sequence listing contained in this .xml file is part of the specification and is hereby incorporated by reference herein in its entirety.

### FIELD OF THE INVENTION

This invention relates to methods to prevent and treat human lipedema including AKR1C1 as a diagnostic and therapeutic target.

### BACKGROUND

Lipedema is a chronic and progressive pathologic condition mainly characterized by an abnormal body fat distribution. It affects extremities with abnormal fat deposition in thighs and legs and in some cases also the arms, while the trunk, hands and feet remain unaffected (Kruppa et al., 2020). For many years it has been grossly misdiagnosed because its similarity with obesity and lymphedema (Fife et al., 2010). Lipedema patients can be distinguished from these two conditions by a series of features such as body disproportion, bilateral symmetry, hematoma tendency and scarce influence of diet, exercise and bariatric surgery. It has been estimated that about 10% of the woman are affected by lipedema worldwide (Buck D W and Herbst K L, 2016). Male cases have been described in very few reports. For this reason, the involvement of sexual hormones in the etiology of the disease has been postulated several times (Torre et al., 2018; Bauer et al., 2019). In line with this hypothesis, manifestations commonly arise in phases of hormonal changes (puberty, pregnancy or menopause) in females (Torre et al., 2018). There is very strong evidence of a genetic base for the condition, since an autosomal dominant hereditary pattern was found in many families (Buso et al., 2019).

While genetic factors apparently regulate subcutaneous adipose tissue distribution, so far, no monogenic cause of non-syndromic primary lipedema have been discovered until now.

Generally, patients with lipedema undergo differential diagnosis from other disorders, and other genes are screened to exclude the patient as having a known diagnosis of another disorder of subcutaneous adipose tissue (ADRA2A, AKT2, ALDH18A1, CIDEC, LIPE, LMNA, MFN2, NSD1, PALB2, PLIN1, POU1F1, PPARG, TBL1XR1) and of localized lipodystrophies (AGPAT2, AKT2, BSCL2, CAV1, CAVIN1 (PTRF), CIDEC, LIPE, LMNA, PLIN1, PPARG, ZMPSTE24).

To date, no direct or specific treatment of the causes of lipedema has been described. Therapies are performed to help relieve symptoms and prevent frustration. When possible, a conservative management is suggested and this include manual lymph drainage, appropriate compression therapy with custom-made, flat-knitted compressive clothing, psychosocial therapy, patient education on self-management, physiotherapy and exercise therapy (such as low impact, cycling, walking or other exercise or movements), dietary counseling and weight management.

Up to now, no effective nutritional treatment has been reported for patients with lipedema. Lipedema fat is resistant to diet therapy. Current dietary approaches are aimed at lowering body weight through a hypocaloric diet, inhibiting systemic inflammation with antioxidant and anti-inflammatory components and reducing water retention (Di Renzo et al., 2021).

In some cases, if symptoms impair quality of life, the potential indication for surgery should be evaluated. Liposuction therapeutic benefit has not yet been evaluated in any randomized, controlled trials. Liposuction can reduce leg circumference, pain, feeling of tightness, tendency to form hematomas, improving quality of life. In highly advanced stages of the disease (i.e. in presence of lymphedema and fibrosis) dermato-fibro-lipectomy may be indicated.

### SUMMARY OF THE INVENTION

This invention provides methods for diagnosing lipedema or identifying agents for treating a patient having lipedema or a predisposition for lipedema. The methods comprise one or more of the following steps:
detecting step to identify variants in the sequence of AKR1C1 gene from gDNA (genomic DNA). Single nucleotide polymorphism (SNP) analysis is also useful for detecting differences between alleles of AKR1C1 genes, that reside within a region of human chromosome 10. Within this region, about 700 known SNPs have been reported to date;
detecting step comprises quantifying mRNA encoding an AKR1C1 isoform in a biological sample (blood, urine and adipose tissue specimens);
detecting increment or reduction of AKR1C1 enzymatic substrate or product (i.e. steroid derivatives and prostaglandins) in a biological sample (blood, urine and adipose tissue specimens) in a lipedema patient compared to controls. The biological sample can be screened with an antibody that specifically binds to AKR1C1 enzymatic substrate or product or the biological sample can be treated or converted by AKR1C1 enzyme;
identifying natural and synthetic molecules capable of modulating AKR1C1 with possible therapeutic effect on lipedema.

Only the identification of AKR1C1 as the first lipedema-associated gene rendered the diagnostic and therapeutic approaches herein described possible. The identification of the gene and its linkage to lipedema opened the way to diagnose and treat the disease of lipedema. Since AKR1C1 is the first gene associated with the molecular diagnosis of non-syndromic lipedema, there are currently no molecular diagnostic alternatives.

Indeed, with their study, the inventors argue in favor of the involvement of AKR1C1 in lipedema (Michelini et al., 2020). AKR1C1 is a gene highly expressed in the subcutaneous tissue and it has been suggested that its activity in the regulation of steroid hormone levels plays an important role in the accumulation of subcutaneous fat depots. The enzyme expressed by this gene, the 20α-hydroxysteroid dehydrogenase (20α-HSD), metabolizes progesterone and causes over production of subcutaneous adipocytes (Blanchette et al., 2005). To date, AKR1C1 has not been implicated in any genetic condition characterized by or including lipedema among its clinical manifestations.

The association may be due to rare genetic variants or common polymorphisms that alter enzymatic function and can also be caused by epigenetic alterations.

In one embodiment of the method for the diagnosis of lipedema and/or for the individuation of treatments thereof according to the invention, the variants of step (i) are detected from gDNA, in particular by single nucleotide polymorphism (SNP) analysis for detecting differences between alleles of AKR1C1 genes, that reside within a region of human chromosome 10, or detected through NGS (Next Generation Sequencing) or Sanger technologies.

In an advantageous embodiment of the method for the diagnosis of lipedema and/or for the individuation of treatments thereof according to the invention, the variants of step (i) are selected from known loss-of-function (LoF) SNPs indicated in table 1 or from a list of selected SNPs as indicated in table 2 or 4. The SNPs can, for example, be selected on the basis of the following criteria: only missense variants; absent in homozygous state; frequency below 0.1%. The selected variants are subsequently preferably studied by functional modelling to verify their impact, for example in terms of binding affinity to certain compounds. This permits to study for one or more particular variants found in a patient the binding affinity to pharmaceutically active compounds, to find the compound that best fits for the particular variant and thus for the patient being affected by this variant.

Preferably, the variants are selected from the group consisting of: c.840C>A (p.Asn280Lys), c.327T>A (p.Aspl09Glu), c.928A>C (p.Ile310Leu), or are selected from c.160T>G (p.Leu54Val), c.162A>T (p.Leu54Phe), c.638T>A (p.Leu213Gln), the p.Leu54 and p. Leu213 variants being particularly preferred. The six variants above are particularly interesting as they have been found in lipedema patients.

In particular, the missense variant p.(Leu213Gln) in AKR1C1, the gene encoding for an aldo-keto reductase catalyzing the reduction of progesterone to its inactive form, 20-α-hydroxyprogesterone, suggests a partial loss-of-function resulting in a slower and less efficient reduction of progesterone to hydroxyprogesterone and an increased subcutaneous fat deposition in variant carriers. The p.(Leu213Gln) variant, to the knowledge of the inventors, is the first one ever identified in a lipedemia family.

Being an inducible gene (Pallai et al., 2010), AKR1C1 expression in the blood can be a marker of the disease. Similarly, urinary and blood plasma or serum metabolites can be used as disease markers and have diagnostic value.

In another embodiment of the method for the diagnosis of lipedema and/or for the individuation of treatments thereof according to the invention, the mRNA of step (ii) or the enzymatic substrate or product or metabolite of step (iii) is detected in a biological sample, in particular in blood, urine and/or adipose tissue specimens.

In a preferred embodiment, the enzymatic substrate or product or metabolite of step (iii) is a steroid derivative or a prostaglandin.

Preferably, the biological sample of step (iii) is screened with an antibody that specifically binds to the AKR1C1 enzymatic substrate or product or metabolite or the biological sample is treated or converted by AKR1C1 enzyme.

Preferably, the enzymatic substrate or product in step (iii) is selected among 20α-hydroxysteroid dehydrogenase (20α-HSD); PGF2α and its derivatives, in particular by measurement of 15-keto-13,14-dihydro-PGF2α, the major metabolite of PGF2α in plasma; or isoprostane 8-iso-Prostaglandin F2α (8-iso-PGF2α).

In a preferred embodiment of the method for the diagnosis of lipedema according to the invention, in step (iii) the levels of at least one of the following metabolites 3α-Hydroxy-5α-pregnan-20-one, 3α-Hydroxy-5β-pregnan-20-one, 3β-Hydroxy-5α-pregnan-20-one, 3β-Hydroxy-5β-pregnan-20-one, 5α-Pregnane-3,20-dione, 5β-Pregnane-3,20-dione, Pregn-4-ene-3,20-dione, 20α-Hydroxy-pregn-4-ene-3-one, 5α-Pregnane-3a,20α-diol, 5β-Pregnane-3a,20α-diol, 5α-Androstan-17β-ol-3-one, 5α-androstane-3a,17β-diol, 21-hydroxy-5α-pregnan-20-one, 3α,21-dihydroxy-5α-pregnan-20-one, Pregnanetriol/17-hydroxypregnanolone, 15-keto-13,14-dihydro-PGF2α, in particular 8-iso-Prostaglandin F2α progesterone and/or 5alpha-dihydrotestosterone is determined in a body fluid.

In another embodiment of the method for the diagnosis of lipedema according to the invention, in step (iii) the ratio (androstanediol^{1.5}×20β-DH-cortisone)/(20β-DH-cortisone+[cortisol×log(estriol)] in a body fluid is determined.

AKR1C1 is a target of natural and synthetic molecules capable of modulating its activity. Benzodiazepines such as medazepam represent a class of non-competitive inhibitors of AKR1C1. Synthetic derivatives of pyrimidine, phthalimide and anthranilic acid potently inhibited AKR1C1 (Brozic et al., 2009). Compounds provided with a core structure of steroid carboxylate and flavones are instead AKR1C1 competitive inhibitors. Among natural compounds, liquiritin has been discovered as a selective and potent AKR1C1 inhibitor capable of reducing the progesterone metabolism in cells (Zeng et al., 2019).

Prostanoids, acting via peroxisome proliferator-activated receptor gamma (PPARγ), a fundamental receptor in fatty acid storage and glucose homeostasis, have been proposed as potent regulators of fat cell differentiation. Indeed, in vitro studies showed that prostaglandin J2 (PGJ2) binds and activates PPARγ acting as a potent adipogenic hormone; inversely, prostaglandin F(2α) (PGF2α), which has PPARγ antagonist properties, is a potent antiadipogenic factor (Quinkler et al., 2006; Volat et al., 2012). Another proof of the involvement of prostaglandins (PG) in the regulation of adipocyte differentiation came from the use of PG analogues as hypotensive agents in the treatment of glaucoma, extensively described in literature reports. Indeed, patients treated with topical therapies based on PG analogues showed periorbital fat changes as an adverse effect. These molecules can directly lead to reduced orbital fat by inhibiting adipogenesis (Taketani et al., 2014). Aldo-keto reductases have been reported as major regulators of white adipose tissue development with antiadipogenic properties supported by PGF2α synthase activity (Quinkler et al., 2006; Volat et al., 2012). Indeed, PGF2α can be synthesized from PGD2 and PGE2 by the enzymes AKR1C (1, 2 and 3) (Quinkler et al., 2006; Dozier et al., 2008) and Akr1b7 (Volat et al., 2012). In vitro studies demonstrated that PGD2 enhances adipocyte differentiation while PGE2 and PGF2α suppress adipogenesis (Miller et al., 1996).

A further aspect of the invention refers to a method of treating and/or preventing of human lipedema in a subject, the method comprising administering or applying to a subject in need thereof a therapeutically effective amount of a compound of natural or synthetic origin, preferably contained in a food supplement, cream or ointment, suitable for modulating the activity of AKR1C1 or of prostaglandins.

In one embodiment of the method of treating and/or preventing of human lipedema in a subject according to the invention, the compound is an inhibitor of AKR1C1 or modulates the catalytic activity of the AKR1C1 enzyme, and comprises at least one of the compounds indicated in table 6, in particular benzodiazepines, such as medazepam, derivatives of pyrimidine, phthalimide and anthranilic acid, competitive inhibitors with a core structure of steroid carboxylate and flavones, and liquiritin. Advantageously, the compound is selected from the group consisting of flavanone, flavone, 3-hydroxyflavone, 5-hydroxyflavone, equilin, diazepam, 20α-hydroxydydrogesterone, coumarin, glycyrrhetinic acid, 7-hydroxyflavone and 3,7-dihydroxyflavone.

In another embodiment of the method of treating and/or preventing of human lipedema in a subject according to the invention, the compound is suitable for modulating prostaglandins and comprises at least one of the compounds indicated in table 7.

Variants of the method of treating and/or preventing of human lipedema in a subject according to the invention foresee, that the step of administering or applying to a subject in need thereof a therapeutically effective amount of a compound of natural or synthetic origin is preceded by a step for the diagnosis of lipedema according to the invention that confirmed the tested person is affected by lipedema. Advantageously, the confirmation of the fact that the tested person is affected by lipedema is obtained by the detection of a biomarker in a body fluid in a concentration exceeding a determined limit value.

A further aspect of the invention relates to a composition for the treatment of human lipedema, in particular in the form of a food supplement, cream or ointment, comprising an inhibitor of AKR1C1 or a compound that modulates the catalytic activity of the AKR1C1 enzyme or of prostaglandins, in particular at least one of the components indicated in tables 6-10.

An additional aspect of the invention relates to a food supplement comprising the composition according to the invention. Another aspect of the invention relates to a cream comprising the composition according to the invention. A final aspect of the invention refers to an ointment comprising the composition according to the invention.

### BRIEF DESCRIPTION OF THE DRAWINGS

FIG. 1 depicts in a molecular simulation the structure of AKR1C1.

FIG. 2 is a plot relating single amino acids of AKR1C1 to their progesterone binding energy.

FIG. 3 depicts a detail of the structure of AKR1C1 and binding situations to progesterone.

FIG. 4 is a plot relating single amino acids of AKR1C1 to their NAPD(H) binding energy.

FIG. 5 illustrates schematically the role of Leu54 in substrate activity for AKR1C1 and AKR1C2.

FIGS. 6(a) and 6(b) depict in a molecular simulation the disrupted interaction between the steroid progesterone and the AKR1C1 enzyme due to the replacement of the same Leu54 by phenylalanine.

FIG. 7 compares in a molecular simulation the cofactor binding contribution of Asn280 and Gln279.

### DETAILED DESCRIPTION OF INVENTION

Diagnostic methods can comprise the sequencing (through next generation sequencing [NGS] or Sanger technologies) of the AKR1C1 gene, or portions of it, or through whole genome and whole exome approaches for the diagnosis of lipedema. Single nucleotide polymorphism (SNP) analysis is also useful for detecting differences between alleles of AKR1C1 genes that reside within a region of human chromosome 10. Within this region, about 700 known SNPs have been reported to date. A list of known loss-of-function (LoF) SNPs is shown in table 1. In addition, a series of SNPs to have effect on protein function and an association with lipedema selected on the basis of the following criteria are listed in table 2: only missense variants; absent in homozygous state; frequency below 0.1%.

TABLE 1
AKR1C1 known LoF variants

| Transcript | Protein | | | Allele |
|---|---|---|---|---|
| Consequence | Consequence | rsID | VEP Annotation | Frequency % |
| c.84 + 1G > T | | rs748912524 | splice_donor_variant | 0.00039896 |
| c.64C > T | p.Gln22* | rs1430171919 | stop_gained | 0.000475064 |
| c.90 + 2T > G | | rs568245058 | splice_donor_variant | 0.029441491 |
| c.100delG | p.Ala34Leufs*2 | rs763666450 | frameshift_variant | 0.000399131 |
| c.134delG | p.Gly45Alafs*30 | rs1138573 | frameshift_variant | 0.000397874 |
| c.172G > T | p.Glu58* | rs1302342979 | stop_gained | 0.000397772 |
| c.81 − 1G > T | | rs530323152 | splice_acceptor_variant | 0.000397779 |
| c.81 − 1G > A | | rs530323152 | splice_acceptor_variant | 0.003580009 |
| c.81 − 1G > C | | rs530323152 | splice_acceptor_variant | 0.000397779 |
| c.196C > T | p.Arg66* | rs201114964 | stop_gained | 0.013793201 |
| c.252 + 2T > C | | rs775284743 | splice_donor_variant | 0.00122379 |
| c.258G > A | p.Trp86* | rs143557246 | stop_gained | 0.000801366 |
| c.271C > T | p.Arg91* | rs139089923 | stop_gained | 0.001775833 |
| c.286C > T | p.Arg96* | rs143132605 | stop_gained | 0.019841832 |
| c.369 + 2T > C | | rs777080970 | splice_donor_variant | 0.008869274 |
| c.394delG | p.Asp132Metfs*44 | rs1188750311 | frameshift_variant | 0.000600478 |
| c.394_397dupGATG | p.Glu133Glyfs*2 | rs1188750311 | frameshift_variant | 0.000600478 |
| c.403G > T | p.Gly135* | rs763837541 | stop_gained | 0.000574132 |
| c.448 − 1G > A | | | splice_acceptor_variant | 0.000397766 |
| c.514C > T | p.Gln172* | rs1220725793 | stop_gained | 0.000397627 |
| c.570 + 1G > A | | rs770791176 | splice_donor_variant | 0.000795494 |
| c.615G > A | p.Trp205* | rs1272520735 | stop_gained | 0.000416171 |
| c.649dupA | p.Ser217Lysfs*58 | rs370014498 | frameshift_variant | 0.000397864 |
| c.667C > T | p.Arg223* | rs781923069 | stop_gained | 0.000796768 |
| c.680 + 1G > A | | rs142084692 | splice_donor_variant | 0.00850732 |
| c.680 + 1G > C | | rs142084692 | splice_donor_variant | 0.003899188 |
| c.680 + 2T > C | | rs757191838 | splice_donor_variant | 0.00079734 |
| c.681 − 1G > A | | rs782472454 | splice_acceptor_variant | 0.000400352 |
| c.681G > A | p.Trp227* | rs782615031 | stop_gained | 0.002134426 |
| c.698delC | p.Pro233Argfs*22 | rs781955346 | frameshift_variant | 0.000712728 |
| c.741delG | p.Lys247Asnfs*8 | rs781870854 | frameshift_variant | 0.001989036 |
| c.748C > T | p.Arg250* | rs782207877 | stop_gained | 0.001988894 |
| c.846 + 1G > A | | rs782167092 | splice_donor_variant | 0.000545756 |
| c.846 + 1G > T | | rs782167092 | splice_donor_variant | 0.003820293 |
| c.910C > T | p.Arg304* | | stop_gained | 0.005656535 |
| c.929 + 1G > A | | rs781944824 | splice_donor_variant | 0.00209389 |
| c.945delT | p.Asn316Ilefs*15 | rs782460823 | frameshift_variant | 0.001235799 |
| c.962delA | p.Asp321Valfs*10 | | frameshift_variant | 0.000818391 |
| c.969T > G | p.Tyr323* | rs201500205 | stop_gained | 0.059779068 |

TABLE 2
AKR1C1 selected variants

| Transcript | | | | |
|---|---|---|---|---|
| Consequence | | | | |
| AKR1C1: | Protein | | | Allele |
| NM 001353.6: | Consequence | rsID | VEP Annotation | Frequency % |
| c.160T + G¹ | p.Leu54Val | rs138675307 | missense_variant | 0.080147155 |
| c.162A + T¹ | p.Leu54Phe | rs14929564 | missense_variant | 0.080147155 |
| c.911G > T² | p.(Arg304Leu) | — | missense_variant | |
| c.381A + T² | p.(Glu127Asp) | — | missense_variant | |
| c.664_665delCAinsAT² | p.(His222Ile) | — | missense_variant | |
| c.664_665delCAinsTC² | p.(His222Ser) | — | missense_variant | |
| c.919_920delACinsGT² | p.(Thr307Val) | — | missense_variant | |
| c.925_926delGAinsCT | p.(Asp309Leu) | — | missense_variant | |
| (p.Asp309Leu)² | | | | |
| c.914A > T² | p.(Tyr305Phe) | — | missense_variant | |
| c.638T > A³ | p.Leu213Gln | rs372782197 | missense_variant | 0.011188627 |
| c.22G > C | p.Val8Leu | rs752938448 | missense_variant | 0.000397735 |
| c.22G > T | p.Val8Leu | rs752938448 | missense_variant | 0.000397735 |
| c.32A > G | p.Asn11Ser | rs1446558895 | missense_variant | 0.000397772 |
| c.5G > A* | p.Gly2Glu | rs1405103238 | missense_variant | 0.000475638 |
| c.82A > G* | p.Met28Val | rs1187727403 | missense_variant | 0.003225598 |
| c.97A > G | p.Lys33Glu | rs1177376359 | missense_variant | 0.000399109 |
| c.104T > C | p.Leu35Ser | rs1174379434 | missense_variant | 0.000397988 |
| c.139C > A | p.Arg47Ser | rs748193660 | missense_variant | 0.000397791 |
| c.163T > C | p.Tyr55His | rs1564314801 | missense_variant | 0.000397725 |
| c.168T > A | p.Asn56Lys | | missense_variant | 0.000397747 |
| c.184G > A | p.Gly62Arg | rs1274415938 | missense_variant | 0.000397829 |
| c.272G > T | p.Arg91Leu | rs375752583 | missense_variant | 0.000399304 |
| c.274C > A | p.Pro92Thr | rs763383627 | missense_variant | 0.000399081 |
| c.290C > G | p.Pro97Arg | rs756379873 | missense_variant | 0.000398594 |
| c.298G > C | p.Glu100Gln | rs1564315232 | missense_variant | 0.000398318 |
| c.338T > C | p.Leu113Pro | rs1344076147 | missense_variant | 0.000398362 |
| c.355C > A | p.Pro119Thr | rs752532298 | missense_variant | 0.000398314 |
| c.392A > T | p.Cys131Ile | rs369662093 | missense_variant | 0.000602736 |
| c.394G > T | p.Asp132Tyr | rs1364894460 | missense_variant | 0.000601214 |
| c.566A > G | p.Asn189Ser | rs771829414 | missense_variant | 0.00039769 |
| c.584A > G* | p.Asp195Gly | rs1407820595 | missense_variant | 0.000417011 |
| c.607C > A* | p.Pro203Thr | rs962503713 | missense_variant | 0.000415866 |
| c.607C > G* | p.Pro203Ala | rs962503713 | missense_variant | 0.000415866 |
| c.575A > C | p.Glu192Ala | rs1564317029 | missense_variant | 0.000399683 |
| c.616T > G | p.Cys206Gly | rs782505662 | missense_variant | 0.00039807 |
| c.698C > T | p.Pro233Leu | rs370027719 | missense_variant | 0.000401068 |
| c.715C > A | p.Pro239Thr | rs1554769975 | missense_variant | 0.000398889 |
| c.755C > G | p.Pro252Arg | rs1303247012 | missense_variant | 0.00039776 |
| c.764T > C | p.Ile255Thr | rs1554770000 | missense_variant | 0.000397782 |
| c.773G > T | p.Arg258Eeu | rs138128200 | missense_variant | 0.000397807 |
| c.787C > G | p.Arg263Gly | rs782766545 | missense_variant | 0.000397842 |
| c.788G > C | p.Arg263Pro | rs535110977 | missense_variant | 0.000397905 |
| c.788G > T | p.Arg263Leu | rs535110977 | missense_variant | 0.003183091 |
| c.797T > C | p.Val266Ala | rs1554770013 | missense_variant | 0.003184105 |
| c.962A > G | p.Asp321Gly | rs1185288451 | missense_variant | 0.000408243 |

*Further studies showed that these variants do not find a unique match between the nucleotide sequence and the amino acid sequence among all the queried databases.
The above variants are, if not stated otherwise, extracted from the following database: https://gnomad.broadinstitute.org/gene/ENSG00000187134?dataset=gnomad_r2_1
¹identified in lipedema patients.
²These variants have been created in a mutagenesis experiment described by Couture et al.
³The enzyme activity parameters described by Couture et al. were used to calculate those of the first variant identified by Michelini et al. in a family with lipedema, p.(Leu213Gln).

The complete sequence of the AKR1C1 gene is well known and documented in literature. The following links take to a database that discloses details about the gene and the whole sequence:
https://www.ensembl.org/Homo_sapiens/Gene/Summary?db=core;g=ENSG00000187134;r=10:496325 3-4983283
https://www.ensembl.org/Homo_sapiens/Transcript/Exons?db=core;g=ENSG00000187134;r=10:4963 253-4983283;t=ENST00000380872

The sequence listing reports the complete sequence of the AKR1C1 gene (Homo sapiens) as SEQ ID NO 1, the corresponding coding sequence (cDNA) as SEQ ID NO 2 and two isoform corresponding proteins as SEQ ID NO 3 and SEQ ID NO 4. The DNA and corresponding protein sequence of the variant c.928A>C (p.(Ile310Leu)) are depicted as SEQ ID NO 5 and SEQ ID NO 6, respectively.

Further details regarding the identification of missense AKR1C1 variants in lipedema patients, sequencing, molecular modelling etc. are described in Michelini S, Chiurazzi P, Marino V, Dell'Orco D, Manara E, Baglivo M, Fiorentino A, Maltese P E, Pinelli M, Herbst K L, Dautaj A, Bertelli M., Aldo-Keto Reductase 1C1 (AKR1C1) as the First Mutated Gene in a Family with Nonsyndromic Primary Lipedema. Int J Mol Sci. 2020 Aug. 29; 21(17):6264. doi: 10.3390/ijms21176264. PMID: 32872468; PMCID: PMC7503355.

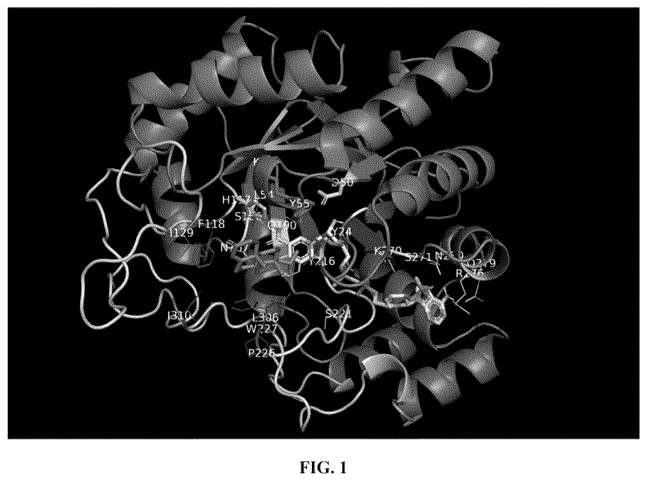

From structural analysis and molecular dynamics it was found that (FIG. 1): Human AKR1C1 three-dimensional structure shows an (αβ)8-barrel motif Two more β-sheets B1 (7-9), B2 (15-17), and two more α-helices, H1(239-248) and H2 (290-298), not taking part in the core barrel structure. Three large loops complete the structure: loop A is located at 117-143, loop B is located at 217-238, and loop C is located at 299-322.

The NADP(H)-binding residues are highly conserved and include Thr23, Asp50, Ser166, Asn167, Gln190, Tyr216, Leu219, Ser221, Arg270, Ser271, Phe272, Arg276, Glu279 and Asn280, which contribute toward the binding affinity and specificity of the cofactor (see FIG. 1). Residues involved in substrate binding are: Tyr24, Leu54, Phe118, Phe129, Thr226, Trp227, Asn306 and Tyr310, while those involved in catalysis are: Asp50, Tyr55, Lys84 and His117 (see FIG. 1).

To describe the interaction of the enzyme with cofactor and substrate in energetic terms, thus to furnish an energy landscape of binding, molecular dynamics simulations were run on the AKR1C1/steroid/NADP(H) ternary complex, and binding energy was calculated as well by use of the MMPBSA (Genheden and Ryde, 2015) method and GROMACS molecular dynamics software (Abraham et al., 2015). The overall energies of binding for the two are (Table 3):

TABLE 3

| | | |
|---|---|---|
| Steroid (STR) | −115.4 kJ/mol | +/− 10.6 kJ/mol |
| NADP(H) (NPD) | −337.6 kJ/mol | +/− 53.9 kJ/mol |

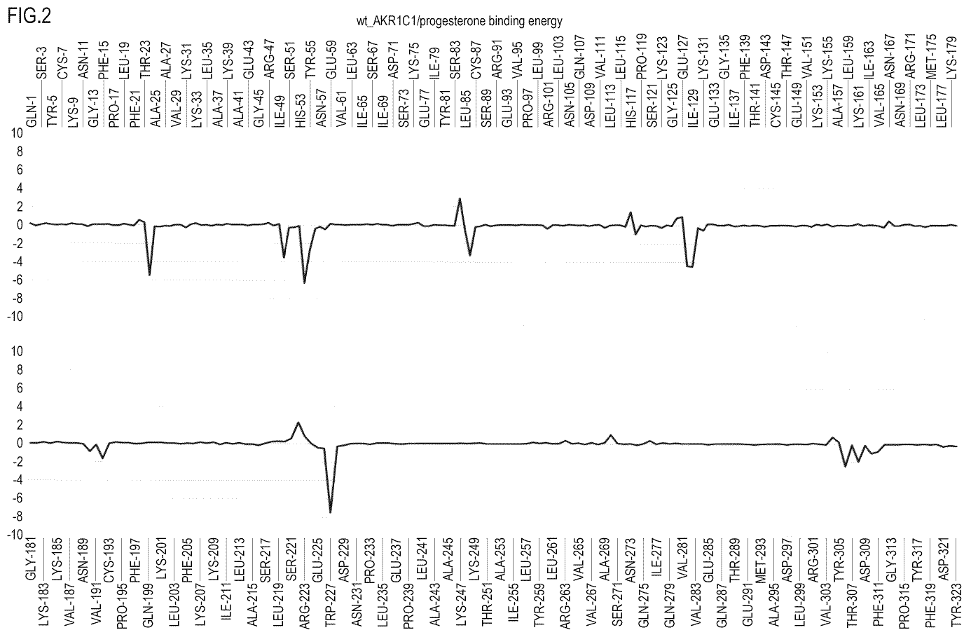

The MMPBSA method also allowed for quantification of the contribution to binding of each amino acid, allowing the impact of an amino acidic missense substitution to be evaluated as follows (see also Table 4). MMPSA profile of STR binding shows three amino acids account for 50% of the binding energy: Tyr24, Leu54, and Trp227; another significant contribution is given by Asp50, Tyr55, Trp86, Val128, Ile129, Leu306, showing an overall hydrophobic nature of the binding (see FIGS. 2 and 3).

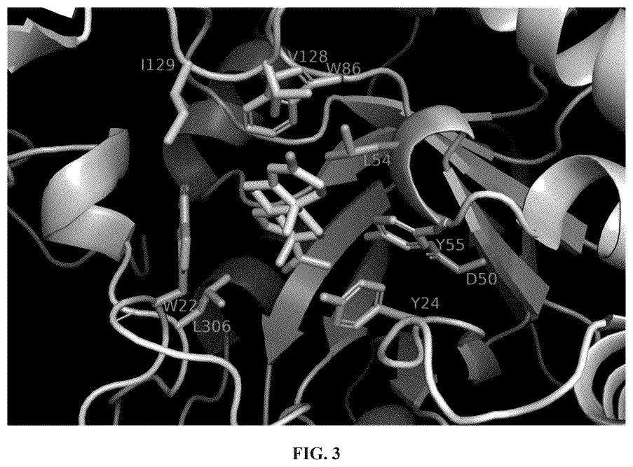

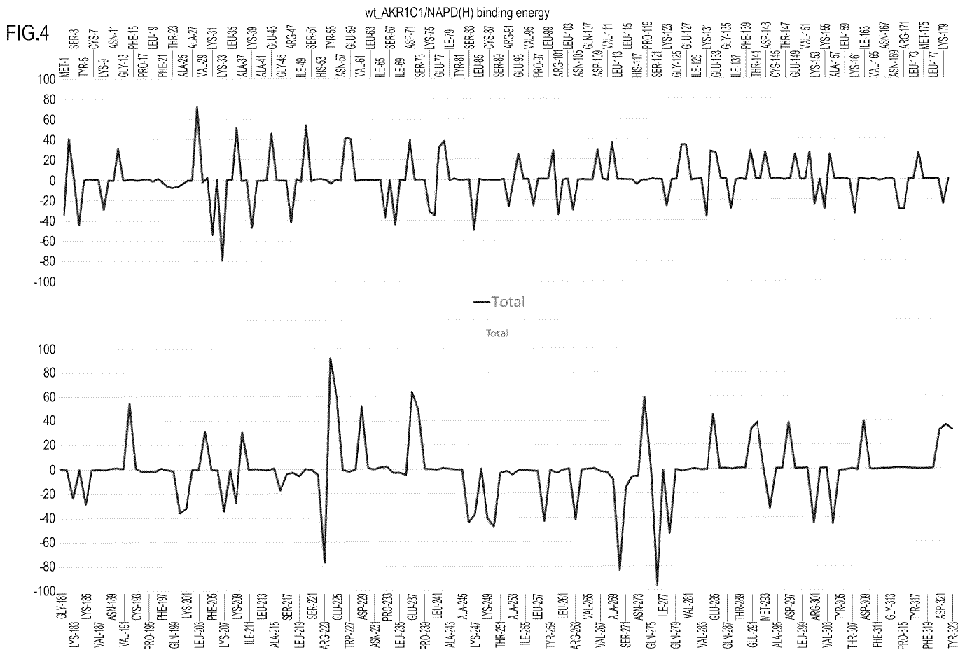

MMPBSA profile of NADP(H) binding is dominated by charge pairs giving prominent repulsion/attraction peaks between charged amino acids of the protein and the phosphate groups of the cofactor. The four most prominent negative binding energy peaks derive from Lys33, His222+Arg223, Lys270, Arg276, all neighboring the phosphate group on the 2′ position of the ribose ring that carries the adenine moiety (see FIG. 4). Such evidence accounts for the significant difference in affinity for NADP(H) vs NAD(H) cofactors in binding AKR enzymes, with the former showing a mid-nanomolar value (100 nM) whereas the latter binds with mid-micromolar affinity (200 mM).

Multiple alignments of protein sequences produce a matrix of aminoacids; by elaborating the columns as vectors, entropy of aminoacidic positions can be calculated according to Shannon, describing the amount of variability through a column in the alignment. The lower the value, the lower the variability accepted by the position. The inventors aligned 120 sequences from the AKR1C family to derive Shannon entropy (Strait & Dewey, 1996) of each position; values for each missense mutation from Table 2 (AKR1C1 selected variants) are reported in Table 4.

In silico mutagenesis of AKR1C1 and molecular dynamics simulations, entropy evaluation, binding energy for cofactor and for substrate allowed for the determination of the structural impact of variants, thus the structural consequence prediction on AKR1C1 that are conducive of loss function for many of the selected mutations in Table 2. Mutations are reported alongside their predicted effect in Table 4.

TABLE 4

| Transcript | | Shannon Entropy | | | |
|---|---|---|---|---|---|
| Consequence | | (natural | Interaction | Interaction | Predicted |
| AKR1C1: | Protein | value/normalized | with | with | structural |
| NM_001353.6: | Consequence | value to 4.32 max) | substrate | cofactor | consequence |
| c.160T > G | p.(Leu54Val)* | 2.11/0.49 | • | | Known to acquire the |
| | | | | | function of AKR1C2^{‡} |
| c.162A > T | p.(Leu54Phe)* | 2.11/0.49 | • | | Disruption of |
| | | | | | substrate binding |
| c.911G > T¹ | p.(Arg304Leu) | 0.61/0.14 | | | Disruption of folding |
| c.381A > T¹ | p.(Glu127Asp) | 1.72/0.40 | | | — |
| c.664_665delCAinsAT¹ | p.(His222Ile) | 1.78/0.41 | | • | Disruption of |
| | | | | | cofactor binding |
| c.664_665delCAinsTC¹ | p.(His222Ser) | 1.78/0.41 | | • | Disruption of |
| | | | | | cofactor binding |
| c.919_920delACinsGT¹ | p.(Thr307Val) | 3.21/0.74 | | | — |
| c.925_926delGAinsCT | p.(Asp309Leu) | 2.95/0.68 | | | — |
| (p.Asp309Leu)¹ | | | | | |
| c.914A > T¹ | p.(Tyr305Phe) | 0.19/0.04 | | | — |
| c.638T > A² | p.(Leu213Gln)* | 0.26/0.06 | | | Disruption of folding |
| c.22G > C | p.(Val8Leu) | 1.07/0.25 | | | — |
| c.22G > T | p.(Val8Leu) | 1.07/0.25 | | | — |
| c.32A > G | p.(Asn111Ser) | 0.47/0.11 | | | — |
| c.97A > G | p.(Lys33Glu) | 1.95/0.45 | | • | Disruption of |
| | | | | | cofactor binding |
| c.104T > C | p.(Leu35Ser) | 2.98/0.69 | | | — |
| c.139C > A | p.(Arg47Ser) | 0.51/0.12 | | | Disruption of folding |
| c.163T > C | p.(Tyr55His) | 0/0 | • | | Disruption of catalysis |
| c.168T > A | p.(Asn56Lys) | 1.88/0.44 | | | — |
| c.184G > A | p.(Gly62Arg) | 0/0 | | | Disruption of folding |
| c.272G > T | p.(Arg91Leu) | 1.17/0.27 | | | Disruption of folding |
| c.274C > A | p.(Pro92Thr) | 0.33/0.08 | | | Disruption of folding |
| c.290C > G | p.(Pro97Arg) | 1.39/0.32 | | | Disruption of folding |
| c.298G > C | p.(Glu100Gln) | 0.14/0.03 | | | — |
| c.338T > C | p.(Leu113Pro) | 0/0 | | | Disruption of folding |
| c.355C > A | p.(Pro119Thr) | 0/0 | | | Disruption of folding |
| c.392A > T | p.(Lys131Ile) | 2.16/0.5 | | | — |
| c.394G > T | p.(Asp132Tyr) | 0.73/0.17 | | | — |
| c.566A > G | p.(Asn189Ser) | 0.14/0.03 | | | Disruption of folding |
| c.575A > C | p.(Glu192Ala) | 0/0 | | | Disruption of folding |
| c.616T > G | p.(Cys206Gly) | 0/0 | | | Disruption of folding |
| c.698C > T | p.(Pro233Leu) | 0.07/0.016 | | | Disruption of folding |
| c.715C > A | p.(Pro239Thr) | 0.12/0.03 | | | Disruption of folding |
| c.755C > G | p.(Pro252Arg) | 0.43/0.10 | | | Disruption of folding |
| c.764T > C | p.(Ile255Thr) | 0.97/0.22 | | | Disruption of folding |
| c.773G > T | p.(Arg258Leu) | 0.07/0.016 | | | Disruption of folding |
| c.787C > G | p.(Arg263Gly) | 0.24/0.06 | | | Disruption of folding |
| c.788G > C | p.(Arg263Pro) | 0.24/0.06 | | | Disruption of folding |
| c.788G > T | p.(Arg263Leu) | 0.24/0.06 | | | Disruption of folding |
| c.797T > C | p.(Val266Ala) | 0.12/0.03 | | | Disruption of folding |
| c.962A > G | p.(Asp321Gly) | 1/0.23 | | | — |
| c.840C > A | p.(Asn280Lys)* | 0.21/0.05 | | • | Disruption of |
| | | | | | cofactor binding |
| c.327T > A | p.(Asp109Glu)* | 0.38/0.09 | | | — |
| c.928A > C³ | p.(Ile310Leu)* | 2.93/0.68 | | | — |

Legend.
*Variants found in lipedema families are marked with an asterisk and a detailed description of structural consequences is reported below;
^{‡}references: (Penning et al., 2019; Hara et al., 1996; Matsuura et al., 1997).
The above variants are, if not stated otherwise, extracted from the following database: https://gnomad.broadinstitute.org/gene/ENSG00000187134?dataset=gnomad_r2_1
¹These variants have been created in a mutagenesis experiment described by Couture et al.
²The enzyme activity parameters described by Couture et al. were used to calculate those of the first variant identified by Michelini et al. in a family with lipedema, p. (Leu213Gln).
³This variant is not described in the above database, the respective DNA and protein sequences are reflected by SEQ ID NO 5 and 6, respectively.

In the following the Applicant reports a detailed descriptions of structural consequences of variants found in lipedema families.

In the inventors' patients, six missense mutations were found, namely Leu54Val, Leu54Phe, Asp109Glu, Asn280Lys, Ile310Leu and Leu213Gln. The effects of such mutations on enzyme folding, stability, and biological activity have been studied with structural biology, and molecular dynamics approach to evaluate their involvement in lipedema development.

Starting with Leu54Val and Leu54Phe, the role of Leu54 in substrate selectivity has been already elucidated (Penning et al., 2019; Hara et al., 1996; Matsuura et al., 1997), and can be summarized as follows (see also FIG. 5). Human AKR1C1 and AKR1C2 differ in that AKR1C1 exhibits 20α-HSD activity, whereas AKR1C2 exhibits 3α-HSD. The two enzymes differ for seven amino acids, and only one is located at the active site at position 54: leucine for C1 and valine for C2. The replacement of Leu54 by the less bulky valine changes the 20α activity to 3α. Consistently, the reverse mutation Val54Leu converts the 3α-HSD into 20α-HSD regarding its activity (Zhang et al., 2014). Evidence that enzymes work in the reduction direction in mammalian cells (Byrns et al., 2010; Byrns et al., 2012; Rizner et al., 2003; Rizner et al., 2006) lead the Leu54Val mutation to hamper the processing of progesterone.

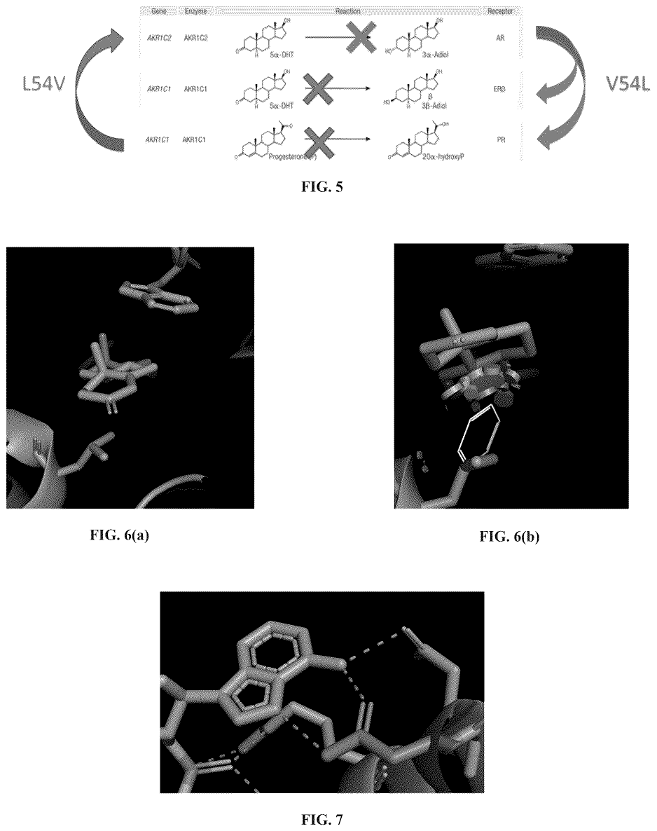

Similarly, the interaction between the steroid and the enzyme is disrupted by the replacement of the same Leu54 by phenylalanine, as shown by the molecular dynamics simulation. In the wildtype, Leu54 and Trp227 play a significant role in binding the steroid by interacting with opposite faces of the polycyclic ring of the ligand and contribute as much as 33% of the overall binding energy. Mutation of Leu54 to Phe, although enhancing the hydrophobic nature of the interaction, introduce a second large, aromatic sidechain in place hampering the ligand entrance in the site and conducive of binding disruption (see FIGS. 6(a) and 6(b)). Indeed, from the molecular dynamics simulations, we noticed that the steroid was unstable, and phenylalanine was pushed back.

Interestingly, phenylalanine is present at position 54 in the wildtype, non-human AKR1C8P, but here the steric hindrance with the opposite amino acid 227 is compensated by the presence of the smaller asparagine. At the same time, the cumbersome tryptophan is ‘shifted’ to position 228. Nonetheless, 1C8 preserve the same 20α-HSD activity of 1C1. As previously mentioned, this may indicate coevolution between positions 54 and 227.

Referring now to Asn280Lys, it can be said that asparagine 280 takes part in cofactor binding; together with Gln279 it is responsible for adenine group binding through a hydrogen bond to the amine group (see FIG. 7). The molecular simulation showed how Asn is the stronger binder of the two. Such finding is also confirmed by the molecular mechanics' energy contributions to the cofactor binding resulting from MMPBSA, showing 6-fold higher interaction energy for Asn280 with respect to Gln279 (17 kJ/mol vs. 3 kJ/mol).

Although such variant involved the replacement of a small side chain with a bulky one, molecular modelling showed how hydrophobic moiety of lysine can be easily accommodated by displacement of water molecules. MD simulation confirmed a small effect is exerted on the protein structure, while the missing H-bond acceptor capability of Lys led to the loss of interaction with the adenine ring, resulting in the aromatic ring flipping away from its position, also because of the attraction of Lys280 to the phosphate group. The optimal binding geometry is then disrupted rather than folding.

On the other hand, Asp109Glu is a small structural change. Furthermore, the conservation at this position is very high, with a relatively low amino acid entropy.

Finally, Ile310Leu is a small structural change since Leu and Ile are isoforms. Furthermore, amino acid entropy is large, implying that position 310 is not conserved throughout evolution.

AKR1C1 is a member of the AKR1C family of enzymes that share a high percentage of amino acid sequence identity (from 84 to 98%). This family catalyzes NADPH dependent oxydoreductions either for the biosynthesis or inactivation of steroid hormones, bile acids and neurosteroids. All AKR1C enzyme catalyze a sequential ordered Bi—Bi substrate enzyme reaction. In particular, AKR1C1 in involved in the “alternative pathway” of androgen biosynthesis inactivating the most potent androgen 5alpha-dihydrotestosterone (5alpha-DHT) to 5alpha-androstane-3beta,17beta-diol, a potent agonist of ERbeta which exerts anti-proliferative effect. Androgens play an important role in regulation of body fat distribution in humans. They exert direct effects on adipocyte differentiation in a depot-specific manner, via the androgen receptor (AR), leading to modulation of adipocyte size and fat compartment expansion. AKR1C1 can also regulate the cellular concentration of allopregnanolone by preventing its formation from progesterone and by catalyzing its inactivation. Indeed, AKR1C1 catalyzes progesterone reaction to form the less potent progestogen 20alpha-hydroxy-4-pregnen-3-one, reduce 5alpha-pregnane-3,20-dione (5alpha-DHP) to form 20alpha-hydroxy-5alpha-pregnan-3-one or 3alpha-hydroxy-5alpha-pregnan-20-one (allopregnanolone) to a less neuroactive 5alpha-pregnane-3alpha,20alpha-diol. AKR1C1 therefore is involved in the inactivation of allopregnanolone, that acts in the central nervous system as positive allosteric modulator of gamma aminobutyric acid receptor A (GABAA). As other enzyme of the family can reduce also 20alpha-hydroxy-5alpha-pregnan-3-one to 5alpha-pregnane-3alpha,20alpha-diol. Progesterone has lipogenic action on adipose tissue by upregulating adipocyte determination and differentiation through 1/sterol regulatory element-binding protein 1c (ADD1/SREBP1c) expression in primary cultured preadipocyte from rat parametrial adipose tissue (Lacasa et al., 2001). ADD1/SREBP1c promotes adipocyte differentiation and gene expression linked to fatty acid metabolism (Kim and Spiegelman, 1996). The levels of progesterone and 5alpha-dihydrotestosterone can be detected in body fluids. Levels of progesterone ranges during normal menstrual cycles from 0 ng/ml (follicular phase) to 28 ng/ml (central luteal phase), values range from 11 to 422 ng/ml during pregnancy, while in post menopause or in males, levels of progesterone are less than 1.2 ng/ml. Levels of 5alpha-DHT range from 250-990 pg/ml in males, from 24-368 in pre menopause females and from 10-181 in post menopause females.

A recent study revealed that the best combination to diagnose polycystic ovary syndrome (PCOS), including up to four steroids, was a ratio comprising androstanediol, estriol, 20βDHcortisone and cortisol accordingly to the following formula: (androstanediol^{1.5}×20β-DH-cortisone)/(20β-DH-cortisone+[cortisol×log(estriol)]. This ratio was significantly increased in PCOS compared to controls at a threshold value of >435 (Dhayat et al., 2018). Considering the activity of the AKR1C1 enzyme, this ratio reasonably has diagnostic value in lipedema.

AKR1C1 is also involved in catalyzing the synthesis of prostaglandins in humans (Dozier et al., 2008). It has been shown that prostaglandin 2 alpha (PGF2α) inhibited adipogenesis by activating at its specific receptor on preadipocytes (Lepak and Serrero, 1995; Taketani et al., 2014). In mice, a decrease in intra-adipose tissue PGF2α levels following Akr1b7 ablation leads to increased adiposity, a phenotype that is reversed by the chronic administration of Cloprostenol, a PGF2α agonist (Volat el al., 2012). PGF2α and its derivatives can therefore be used as molecular diagnostic/prognostic markers and therapeutic agents also in lipedema. PGF2α can be reliably quantified by measurement of 15-keto-13,14-dihydro-PGF2α, the major metabolite of PGF2α in plasma (Helmersson et al., 2005). The isoprostane 8-iso-Prostaglandin 1F2α (8-iso-PGF2α), a prostaglandin-like molecule, is a quantitative ROS biomarker used to measure oxidative stress in vivo which correlates positively with BMI, intra-abdominal fat and waist circumference (Milne et al., 2015; Jia et al., 2019). Both molecules can be easily quantified in different body fluids such as plasma, serum or urine.

A list of AKR1C1 metabolites for use in diagnostics is reported in table 5.

TABLE 5
AKR1C1 metabolites for use in diagnostics

| Molecule | Common name |
|---|---|
| 3α-Hvdroxy-5α-pregnan-20-one | Allopregnanolone (allo) |
| 3α-Hydroxy-5β-pregnan-20-one | Pregnanolone (preg) |
| 3β-Hvdroxy-5α-pregnan-20-one | Isopregnanolone (iso) |
| 3β-Hydroxy-5β-pregnan-20-one | Epipregnanolone (epi) |
| 5α-Pregnane-3,20-dione | 5α-Dihydroprogesterone (5α-DHP) |
| 5β-Pregnane-3,20-dione | 5β-Dihydroprogesterone (5β-DHP) |
| Pregn-4-ene-3,20-dione | Progesterone (P) |
| 20α-Hydroxy-pregn-4-ene-3-one | 20α-dihydroprogesterone (20α-OHP) |
| 5α-Pregnane-3α,20α-diol | Allopregnanediol |
| 5β-Pregnane-3α,20α-diol | Pregnanediol |
| 5α-Androstan-17β-ol-3-one | 5α-Dihydrotestosterone (5α-DHT) |
| 5α-androstane-3α,17β-diol | 3α-Androstanediol (3α-Adiol) |
| 21-hydroxy-5α-pregnan-20-one | 5α-Dihydrodeoxycorticosterone (5αDHDOC) |
| 3a,21-dihydroxy-5α-pregnan-20-one | 3α,5α-Tetrahydrodeoxycorticosterone |
| Pregnanetriol/17-hydroxypregnanolone | (alloTHDOC) (P3)/(17HP) |
| 15-keto-13,14-dihydro-PGF2α | PGFM |
| 8-iso-Prostaglandin F2α | 8-iso-PGF2α |

In the literature, a number of natural and synthetic compounds are known to exert a modulatory action on the key human progesterone-metabolizing enzyme, AKR1C1.

A list of compounds for treatments for lipedema comprising the use of natural molecules or chemicals that modulate the catalytic activity of the AKR1C1 enzyme are shown in table 6.

TABLE 6
Natural and synthetic compounds that modulate AKR1C1

| | | Enzyme activity |
|---|---|---|
| Compound | Main sources | (inhibition/activation) |
| 2,3-dimethoxynaphthalene-1,4-dione (DMNQ) | Synthetic | activation |
| 20α-hydroxydydrogesterone | Synthetic | inhibition |
| 3,5-dichlorosalycilic acid | Synthetic | inhibition |
| 3,5-diiodosalycilic acid | Synthetic | inhibition |
| 3,7-dihydroxyflavone | Synthetic | inhibition |
| 3-bromo-5-phenylsalicylic acid | Synthetic | inhibition |
| 3-Hihydroxy flavone | Synthetic | inhibition |
| 5-Hihydroxy flavone | Synthetic | inhibition |
| 5,7-Dihydroxyflavone | Passiflora coerulea | inhibition |
| 5-Metoxy flavone | Synthetic | inhibition |
| 7-Hydroxy flavone | Synthetic | inhibition |
| Abietic acid | Pine wood | inhibition |
| AKR1C1 Inhibitor, 5-PBSA | Synthetic | inhibition |
| AKR1C1-IN-1 | Synthetic | inhibition |
| Apigenin | Snapdragon, chamomille | inhibition |
| Benzodiazepines (diazepam, | Synthetic | inhibition |
| medazepam, estazolam, flunitrazepam, | | |
| nitrazepam, cloxazolam, bromazepam, | | |
| oxazolam and oxazepam) | | |
| Biochanin A | Red clover, soy, alfalfa sprouts, | inhibition |
| | peanuts, chickpea (Cicer | |
| | arietinum) and in other legumes | |
| Chrysin | Scutellaria baicalensis | inhibition |
| Coumarin | Woodruff, vanilla, lavender oil, | inhibition |
| | tonka bean, minor constituent in | |
| | cherries, strawberries, apricots | |
| Coumestrol | Soybeans, brussels sprouts, | inhibition |
| | spinach and a variety of legumes, | |
| | clover, Kala Chana, Alfalfa sprouts | |
| Cyclopentanone | Synthetic | inhibition |
| Curcumin | Curcuma longa | Unknown |
| Daidzein | Soybeans, beer | inhibition |
| Diethylstilbestrol | Synthetic | inhibition |
| Dydrogesterone | Synthetic | inhibition |
| Equilin | Horse estrogen; estrogen | inhibition |
| | replacement therapy | |
| Ethacrynic acid | Synthetic | activation |
| Flavanone | yellow/red fruits, vegetables | inhibition |
| Flavone | yellow/red fruits, vegetables | inhibition |
| Genistein | Soybeans, beer | inhibition |
| Glycyrrhetinic acid | Licorice | inhibition |
| Hydrogen peroxide | Synthetic | activation |
| Kaempferol | Tea, grapes, berries and | inhibition |
| | cruciferous vegetables | |
| Liquiritin | Licorice | inhibition |
| Luteolin | Parsley, artichoke, basil, celery | inhibition |
| Mangosteen extract | Mangosteen | inhibition |
| Medroxyprogesterone acetate | Synthetic | inhibition |
| Methyl jasmonate | Derived from jasmonic acid as | inhibition |
| | found in many plants | |
| Naringenin | Grapefruit | inhibition |
| Nonsteroidal Anti-Inflammatory Drugs | Synthetic | inhibition |
| (mefenamic acid, indomethacin, | | |
| celecoxib, diclofenac, naproxen, | | |
| ibuprofen, ketoprofen, paracetamol, | | |
| acetylsalicylic acid, etodolac, 3- | | |
| phenoyxbenzoic acid, sulindac, | | |
| meclofenamic acid, zomepirac, | | |
| Norethinodrone | Synthetic | inhibition |
| Quercetin | Chamomille, red onions, apples, | inhibition |
| | tea, endive | |
| Resveratrol | Skins of certain red, grapes, in | inhibition |
| | peanuts, blueberries, pines, roots | |
| | and stalks of knotweed | |
| Steroidal Inhibitors | Synthetic | inhibition |
| (medroxyprogesterone acetate, | | |
| bethamethasone, steroidal lactones, | | |
| cholanic acid derivatives | | |
| t-butylhydroquinone | Synthetic | activation |
| Tamoxifen | Synthetic | inhibition |
| Wagonin | Scutellaria baicalensis | inhibition |
| Zearalenone | Mold-infected grain and feeds | inhibition |

PGE2 and PGF2α and its analogue (viprostol, latanoprost, isopropyl unoprostone, bimatoprost) can exhibit antiadipogenic properties. Some active constituents from Chinese herbs as ricinoleic acid, acteoside, amentoflavone, quercetin-3-O-rutinoside and hinokiflavone were predicted to be prostaglandin D2 synthase (PTGDS) inhibitors (Fong et al., 2015). Inversely, other natural supplements such as chlorella and green tea are proposed be used to decrease PGE2 and PGF2α levels (Koeberle et al., 2009; Haidari et al., 2018).

A list of compounds for treatments of lipedema comprising the use of natural molecules or chemicals that modulate prostaglandins are shown in table 7.

TABLE 7
Natural and synthetic compounds that modulate prostaglandins

| Compound | Main sources | Activity |
|---|---|---|
| Acteoside | Rehmannia glutinosa | PTGDS inhibitors |
| Amentoflavone | Biota orientalis | PTGDS inhibitors |
| Chlorella | Chlorella | decrease PGE2 and PGF2α levels |
| Green tea | Green tea | decrease PGE2 levels |
| Hinokiflavone | Platycladus orientalis | PTGDS inhibitors |
| Quercetin-3-O-rutinoside | Platycladus orientalis | PTGDS inhibitors |
| Ricinoleic acid | Ricinus communis | PTGDS inhibitors |
| Sennosides | Cassia species | increase PGE2 formation |
| Viprostol, latanoprost, isopropyl | Syntetic | PGF2α analogues |
| unoprostone, bimatoprost | | |

Natural and synthetic compounds that modulate AKR1C1 and listed in Table 6 were submitted to molecular docking procedure by using Autodock Vina 1.2 with the following parameters: AKR1C1 and NADPH coordinate from PDB entry 1MRQ; amino acids Tyr24, Leu54 and Trp227 set as flexible sidechain: docking box set centered at x=4.29 y=33.9 z=17.06 with size x=17.39 y=11.16 z=12.67, vina scoring function. Results are reported as binding affinity in Kcal/mol (the lowest, the better) in Table 8. Taking 2 Kcal/mol as the common threshold for binding energy significance, we have 11 top compounds (in bold); significantly 6 out of 11 are simple flavones/flavonones (in bold and italics). The double stacking interaction of B ring with Tyr24 phenol and Trp227 indole rings is the driving force of the interaction.

TABLE 8
Docking analysis of natural and synthetic
compounds that modulate AKR1C1

| | Binding affinity |
|---|---|
| Compound | (Kcal/mol) |
| Flavanone | −17.45 |
| Flavone | −17.25 |
| 3-Hydroxyflavone | −16.12 |
| 5-Hydroxyflavone | −16.09 |
| Equilin | −15.84 |
| Diazepam | −15.70 |
| 20α-Hydroxydydrogesterone | −15.69 |
| Coumarin | −15.58 |
| Glycyrrhetinic Acid | −15.57 |
| 7-Hydroxyflavone | −15.54 |
| 3,7-Dihydroxyflavone | −15.48 |
| Coumestrol | −15.20 |
| Apigenin | −15.16 |
| Flurbiprofen | −15.06 |
| Abietic Acid | −15.04 |
| Mefenamic Acid | −15.03 |
| Beta-Mangostin | −15.00 |
| Cholanic Acid | −14.98 |
| Alpha-Mangostin | −14.96 |
| 5,7-Dyhydroxyflavone | −14.94 |
| Naringenin | −14.93 |
| Ketoprofen | −14.91 |
| Naproxen | −14.70 |
| Luteolin | −14.70 |
| Quercetin | −14.65 |
| Gamma-Mangostin | −14.62 |
| Norethindrone | −14.60 |
| Betamethasone | −14.51 |
| Biochanin A | −14.43 |
| Oxazolam | −14.42 |
| Medazepam | −14.42 |
| 3-Bromo,5-Phenylsalicylic Acid | −14.32 |
| 5-Pbsa | −14.32 |
| Genistein | −14.28 |
| Liquiritin | −14.18 |
| Meclofenamic Acid | −14.17 |
| Sulindac | −14.09 |
| 5-Methoxyflavone | −14.06 |
| Zearalenone | −13.98 |
| Nitrazepam | −13.96 |
| Estazolam | −13.85 |
| Kaempferol | −13.85 |
| Spironolactone | −13.75 |
| Wagonin | −13.74 |
| Bromazepam | −13.73 |
| Indomethacin | −13.71 |
| Daidzein | −13.68 |
| Oxazepam | −13.66 |
| Paracetamol | −13.66 |
| Resveratrol | −13.50 |
| Medroxyprogesterone Acetate | −13.46 |
| 2,3-Dimethoxynaphthalene-1,4-Dione | −13.43 |
| Cloxazolam | −13.40 |
| Medroxyprogesterone Acetate | −13.33 |
| Diethylstilbestrol | −13.31 |
| Ibuprofen | −13.23 |
| Cyclopentanone | −13.20 |
| Zomepirac | −13.03 |
| 3,5-Dichlorosalicylic Acid | −12.97 |
| Flunitrazepam | −12.93 |
| Tamoxifen | −12.89 |
| Hydroxytyrosol | −12.75 |
| T-Butylhydroquinone | −12.60 |
| Curcumin | −12.52 |
| Ethacrynic Acid | −12.37 |
| Methyl Jasmonate | −11.84 |
| 3,5-Diiodosalicylic Acid | −11.76 |

The analysis has been repeated for AKR1C1 mutant Leu54Val by using the same parameters (Table 9). Such mutation is known to convert enzymatic activity of AKR1C1 into that of AKR1C2, which might eliminate androgen inhibitory effects on adipogenesis favouring progression of adipogenesis (Kiani et al., 2021), thus selective targeting of such mutation would modulate its possible effect on fat deposition. Again, flavones are among the favorites, but with lower affinity and competing with natural steroids like equilin or with large pentacyclic molecules glycyrrhetinic acid; this is due to the lower steric hindrance of valine vs. leucine resulting in less selective active site.

TABLE 9
Docking analysis of natural and synthetic compounds
interacting with AKR1C1 mutant Leu54Val

| | Binding affinity |
|---|---|
| Compound | (Kcal/mol) |
| Glycyrrhetinic Acid | −14.41 |
| Equilin | −14.22 |
| Flavone | −14.18 |
| Flavanone | −14.11 |
| 3-Hydroxyflavone | −14.10 |
| Abietic Acid | −13.91 |
| Coumestrol | −13.63 |
| Nitrazepam | −13.59 |
| Betamethasone | −13.52 |
| 5-Hydroxyflavone | −13.49 |
| 20α-Hydroxydydrogesterone | −13.46 |
| Diazepam | −13.42 |
| Cholanic Acid | −13.41 |
| Alpha-Mangostin | −13.41 |
| Gamma-Mangostin | −13.38 |
| Beta-Mangostin | −13.34 |
| Estazolam | −13.29 |
| Genistein | −13.24 |
| Sulindac | −13.08 |
| 5-Pbsa | −13.04 |
| 3-Bromo-5-Phenylsalicylic Acid | −13.04 |
| 3,7-Dihydroxyflavone | −13.00 |
| Oxazepam | −12.93 |
| Norethindrone | −12.84 |
| 7-Hydroxyflavone | −12.84 |
| Naringenin | −12.84 |
| Daidzein | −12.80 |
| 5-Methoxyflavone | −12.72 |
| 5,7-Dyhydroxyflavone | −12.62 |
| Flunitrazepam | −12.61 |
| Bromazepam | −12.59 |
| Celecoxib | −12.59 |
| Zomepirac | −12.58 |
| Apigenin | −12.57 |
| Luteolin | −12.56 |
| Kaempferol | −12.51 |
| Quercetin | −12.47 |
| Medroxyprogesterone-Acetate | −12.41 |
| Medroxyprogesterone-Acetate | −12.41 |
| Coumarin | −12.40 |
| Wagonin | −12.39 |
| Zearalenone | −12.35 |
| Indomethacin | −12.30 |
| Meclofenamic Acid | −12.24 |
| Biochanin-A | −12.17 |
| Flurbiprofen | −12.11 |
| Ketoprofen | −12.07 |
| Liquiritin | −12.06 |
| Diclofenac | −12.05 |
| Naproxen | −12.04 |
| Mefenamic Acid | −11.94 |
| Medazepam | −11.69 |
| 3-Phenoxybenzoic Acid | −11.55 |
| Resveratrol | −11.50 |
| Oxazolam | −11.46 |
| Cloxazolam | −11.42 |
| Diethylstilbestrol | −11.39 |
| Spironolactone | −11.33 |
| Etodolac | −11.33 |
| Tamoxifen | −11.18 |
| Hydroxytyrosol | −11.08 |
| Methyl-Jasmonate | −10.83 |
| T-Butylhydroquinone | −10.79 |
| Cyclopentanone | −10.69 |
| Curcumin | −10.61 |
| Ethacrynic Acid | −10.57 |
| 2,3-Dimethoxynaphthalene-1,4-Dione | −10.42 |
| Ibuprofen | −10.24 |
| Paracetamol | −10.15 |
| Acetylsalicylic Acid | −10.12 |
| 3,5-Diiodosalicylic Acid | −10.02 |
| 3,5-Dichlorosalicylic Acid | −9.69 |

AKR1C1 Leu54Phe mutant is the other variant affecting substrate binding site accessibility presently analyzed. Oppositely but coherently with Leu54Val flavones are the tighter binders due to the incremented steric hindrance of phenylalanine which is able to stacking interact with A/C rings of the binder (Table 10).

TABLE 10
Docking analysis of natural and synthetic compounds
interacting with AKR1C1 mutant Leu54Val

| | Binding affinity |
|---|---|
| Compound | (Kcal/mol) |
| Flavone | −18.35 |
| Flavanone | −18.21 |
| Medroxyprogesterone-Acetate | −18.04 |
| Cholanic Acid | −17.96 |
| Equilin | −17.81 |
| Estazolam | −17.48 |
| 5-Hydroxyflavone | −17.29 |
| Nitrazepam | −17.29 |
| Diazepam | −17.11 |
| 7-Hydroxyflavone | −16.94 |
| 20α-Hydroxydydrogesterone | −16.81 |
| Zearalenone | −16.76 |
| Spironolactone | −16.45 |
| 3-Hydroxyflavone | −16.42 |
| Medazepam | −16.35 |
| Cloxazolam | −16.33 |
| Norethindrone | −16.30 |
| Sulindac | −16.24 |
| Glycyrrhetinic Acid | −16.17 |
| Ketoprofen | −16.15 |
| 5,7-Dyhydroxyflavone | −16.13 |
| 5-Pbsa | −16.05 |
| 3-Bromo-5-Phenylsalicylic Acid | −16.05 |
| Betamethasone | −15.93 |
| Daidzein | −15.91 |
| Gamma-Mangostin | −15.77 |
| Naproxen | −15.72 |
| 3-7-Dihydroxyflavone | −15.72 |
| Coumestrol | −15.70 |
| Apigenin | −15.68 |
| T-Butylhydroquinone | −15.66 |
| Luteolin | −15.60 |
| Naringenin | −15.56 |
| Genistein | −15.54 |
| Celecoxib | −15.51 |
| Bromazepam | −15.51 |
| Resveratrol | −15.46 |
| Oxazolam | −15.46 |
| Liquiritin | −15.46 |
| Abietic Acid | −15.44 |
| Coumarin | −15.43 |
| Alpha-Mangostin | −15.27 |
| 3-Phenoxybenzoic Acid | −15.24 |
| Etodolac | −15.23 |
| 3,5-Dichlorosalicylic Acid | −15.23 |
| Biochanin-A | −15.22 |
| Flurbiprofen | −15.21 |
| Diethylstilbestrol | −15.12 |
| Wagonin | −15.09 |
| Oxazepam | −15.02 |
| Flunitrazepam | −15.01 |
| Kaempferol | −14.96 |
| 5-Methoxyflavone | −14.87 |
| Mefenamic Acid | −14.82 |
| Zomepirac | −14.57 |
| Beta-Mangostin | −14.56 |
| 3,5-Diiodosalicylic Acid | −14.46 |
| Acetylsalicylic Acid | −14.34 |
| Methyl-Jasmonate | −14.09 |
| Paracetamol | −13.97 |
| Diclofenac | −13.92 |
| Quercetin | −13.88 |
| Hydroxytyrosol | −13.86 |
| Indomethacin | −13.80 |
| Meclofenamic Acid | −13.78 |
| 2,3-Dimethoxynaphthalene-1,4-Dione | −13.73 |
| Ibuprofen | −13.58 |
| Cyclopentanone | −13.29 |
| Curcumin | −13.00 |
| Tamoxifen | −12.89 |
| Ethacrynic Acid | −12.19 |

The molecules of tables 9 and 10 have been analyzed considering the interaction with two specific variants, both on nucleotide 54. For every substance indicated in table 8, it is possible to identify through a study determining the affinity to AKR1C1 the most efficient one for a patient with a specific variant, as done for a patient with a variant on nucleotide 54.

### REFERENCES

Abraham M J, Murtola T, Schulz R, Páll S, Smith J C, Hess B, and Lindahl E. GROMACS: High performance molecular simulations through multi-level parallelism from laptops to supercomputers. SoftwareX. 2015; 1-2:19-25. doi.org/10.1016/j.softx.2015.06.001.
Bauer A T, von Lukowicz D, Lossagk K, Aitzetmueller M, Moog P, Cerny M, Erne H, Schmauss D, Duscher D, Machens H G. New Insights on Lipedema: The Enigmatic Disease of the Peripheral Fat. Plast Reconstr Surg. 2019 December; 144(6):1475-1484. doi: 10.1097/PRS.0000000000006280. PMID: 31764671.
Blanchette S, Blouin K, Richard C, Dupont P, Luu-The V, Tchernof A. Expression and activity of 20alpha-hydroxysteroid dehydrogenase (AKR1C1) in abdominal subcutaneous and omental adipose tissue in women. J Clin Endocrinol Metab. 2005 January; 90(1):264-70. doi: 10.1210/jc.2004-0583. Epub 2004 Oct. 19. PMID: 15494462.
Brozic P, Cesar J, Kovac A, Davies M, Johnson A P, Fishwick C W, Lanisnik Rizner T, Gobec S. Derivatives of pyrimidine, phthalimide and anthranilic acid as inhibitors of human hydroxysteroid dehydrogenase AKR1C1. Chem Biol Interact. 2009 Mar. 16; 178(1-3):158-64. doi: 10.1016/j.cbi.2008.10.019. Epub 2008 Oct. 22. PMID: 19007763.
Buck D W 2nd, Herbst K L. Lipedema: A Relatively Common Disease with Extremely Common Misconceptions. Plast Reconstr Surg Glob Open. 2016 Sep. 28; 4(9):e1043. doi: 10.1097/GOX.0000000000001043. PMID: 27757353; PMCID: PMC5055019.
Buso G, Depairon M, Tomson D, Raffoul W, Vettor R, Mazzolai L. Lipedema: A Call to Action! Obesity (Silver Spring). 2019 October; 27(10):1567-1576. doi: 10.1002/oby.22597. PMID: 31544340; PMCID: PMC6790573.
Byrns M C, Duan L, Lee S H, Blair I A, Penning T M. Aldo-keto reductase 1C3 expression in MCF-7 cells reveals roles in steroid hormone and prostaglandin metabolism that may explain its over-expression in breast cancer. J Steroid Biochem Mol Biol. 2010 Feb. 15; 118(3):177-87. doi: 10.1016/j.jsbmb.2009.12.009. Epub 2009 Dec. 28. PMID: 20036328; PMCID: PMC2819162.
Byrns M C, Mindnich R, Duan L, Penning T M. Overexpression of aldo-keto reductase 1C3 (AKR1C3) in LNCaP cells diverts androgen metabolism towards testosterone resulting in resistance to the 5α-reductase inhibitor finasteride. J Steroid Biochem Mol Biol. 2012 May; 130(1-2):7-15. doi: 10.1016/j.jsbmb.2011.12.012. Epub 2012 Jan. 12. PMID: 22265960; PMCID: PMC3319280.
Couture J F, Legrand P, Cantin L, Luu-The V, Labrie F, Breton R. Human 20alpha-hydroxysteroid dehydrogenase: crystallographic and site-directed mutagenesis studies lead to the identification of an alternative binding site for C21-steroids. J Mol Biol. 2003; 331(3):593-604. doi:10.1016/s0022-2836(03)00762-9.
Dhayat N A, Marti N, Kollmann Z, Troendle A, Bally L, Escher G, Grössl M, Ackermann D, Ponte B, Pruijm M, Müller M, Vogt B, Birkhäuser M H, Bochud M, Flück C E; members of the SKIPOGH Study Group. Urinary steroid profiling in women hints at a diagnostic signature of the polycystic ovary syndrome: A pilot study considering neglected steroid metabolites. PLoS One. 2018 Oct. 11; 13(10):e0203903. doi: 10.1371/journal.pone.0203903. PMID: 30308019; PMCID: PMC6181287.
Di Renzo L, Cinelli G, Romano L, Zomparelli S, Lou De Santis G, Nocerino P, Bigioni G, Arsini L, Cenname G, Pujia A, Chiricolo G, De Lorenzo A. Potential Effects of a Modified Mediterranean Diet on Body Composition in Lipoedema. Nutrients. 2021 Jan. 25; 13(2):358. doi: 10.3390/nu13020358. PMID: 33504026.
Dozier B L, Watanabe K, Duffy D M. Two pathways for prostaglandin F2 alpha synthesis by the primate periovulatory follicle. Reproduction. 2008 July; 136(1):53-63. doi: 10.1530/REP-07-0514. Epub 2008 Apr. 4. PMID: 18390687; PMCID: PMC2656351.
Fife C E, Maus E A, Carter M J. Lipedema: a frequently misdiagnosed and misunderstood fatty deposition syndrome. Adv Skin Wound Care. 2010 February; 23(2):81-92; quiz 93-4. doi: 10.1097/01.ASW.0000363503.92360.91. PMID: 20087075.
Fong P, Tong H H, Ng K H, Lao C K, Chong C I, Chao C M. In silico prediction of prostaglandin D2 synthase inhibitors from herbal constituents for the treatment of hair loss. J Ethnopharmacol. 2015 Dec. 4; 175:470-80. doi: 10.1016/j.jep.2015.10.005. Epub 2015 Oct. 9. PMID: 26456343.
Genheden S, Ryde U. The MM/PBSA and MM/GBSA methods to estimate ligand-binding affinities. Expert Opin Drug Discov. 2015 May; 10(5):449-61. doi: 10.1517/17460441.2015.1032936. Epub 2015 Apr. 2. PMID: 25835573; PMCID: PMC4487606.
Haidari F, Homayouni F, Helli B, Haghighizadeh M H, Farahmandpour F. Effect of chlorella supplementation on systematic symptoms and serum levels of prostaglandins, inflammatory and oxidative markers in women with primary dysmenorrhea. Eur J Obstet Gynecol Reprod Biol. 2018 October; 229:185-189. doi: 10.1016/j.ejogrb.2018.08.578. Epub 2018 Aug. 27. PMID: 30205315.
Hara A, Matsuura K, Tamada Y, Sato K, Miyabe Y, Deyashiki Y, Ishida N. Relationship of human liver dihydrodiol dehydrogenases to hepatic bile-acid-binding protein and an oxidoreductase of human colon cells. Biochem J. 1996 Jan. 15; 313 (Pt 2)(Pt 2):373-6. doi: 10.1042/bj3130373. PMID: 8573067; PMCID: PMC1216918.
Helmersson J, Arnlöv J, Vessby B, Larsson A, Alfthan G, Basu S. Serum selenium predicts levels of F2-isoprostanes and prostaglandin F2alpha in a 27 year follow-up study of Swedish men. Free Radic Res. 2005 July; 39(7):763-70. doi: 10.1080/10715760500108513. PMID: 16036356.
Jia X J, Liu L X, Tian Y M, Wang R, Lu Q. The correlation between oxidative stress level and intra-abdominal fat in obese males. Medicine (Baltimore). 2019 February; 98(7):e14469. doi: 10.1097/MD.0000000000014469. PMID: 30762765; PMCID: PMC6408049.
Kiani A K, Mor M, Bernini A, Fulcheri E, Michelini S, Herbst K L, Buffelli F, Belgrado J P, Kaftalli J, Stuppia L, Dautaj A, Dhuli K, Guda T, Manara E, Maltese P E, Michelini S, Chiurazzi P, Paolacci S, Ceccarini M R, Beccari T, Bertelli M. Steroid-converting enzymes in human adipose tissues and fat deposition with a focus on AKR1C enzymes. Eur Rev Med Pharmacol Sci. 2021 December; 25(1 Suppl):23-32. doi: 10.26355/eurrev_202112_27330. PMID: 34890031.
Kim J B, Spiegelman B M. ADD1/SREBP1 promotes adipocyte differentiation and gene expression linked to fatty acid metabolism. Genes Dev. 1996 May 1; 10(9):1096-107. doi: 10.1101/gad.10.9.1096. PMID: 8654925.
Koeberle A, Bauer J, Verhoff M, Hoffmann M, Northoff H, Werz O. Green tea epigallocatechin-3-gallate inhibits microsomal prostaglandin E(2) synthase-1. Biochem Biophys Res Commun. 2009 Oct. 16; 388(2):350-4. doi: 10.1016/j.bbrc.2009.08.005. Epub 2009 Aug. 6. PMID: 19665000.
Kruppa P, Georgiou I, Biermann N, Prantl L, Klein-Weigel P, Ghods M. Lipedema-Pathogenesis, Diagnosis, and Treatment Options. Dtsch Arztebl Int. 2020 Jun. 1; 117(22-23):396-403. doi: 10.3238/arztebl.2020.0396. PMID: 32762835; PMCID: PMC7465366.
Lacasa D, Le Liepvre X, Ferre P, Dugail I. Progesterone stimulates adipocyte determination and differentiation 1/sterol regulatory element-binding protein 1c gene expression. potential mechanism for the lipogenic effect of progesterone in adipose tissue. J Biol Chem. 2001 Apr. 13; 276(15):11512-6. doi: 10.1074/jbc.M008556200. Epub 2001 Jan. 16. PMID: 11278421.
Lepak N M, Serrero G. Prostaglandin F2 alpha stimulates transforming growth factor-alpha expression in adipocyte precursors. Endocrinology. 1995 August; 136(8):3222-9. doi: 10.1210/endo.136.8.7628355. PMID: 7628355.
Matsuura K, Deyashiki Y, Sato K, Ishida N, Miwa G, Hara A. Identification of amino acid residues responsible for differences in substrate specificity and inhibitor sensitivity between two human liver dihydrodiol dehydrogenase isoenzymes by site-directed mutagenesis. Biochem J. 1997 Apr. 1; 323 (Pt 1)(Pt 1):61-4. doi: 10.1042/bj3230061. PMID: 9173902; PMCID: PMC1218315.
Michelini S, Chiurazzi P, Marino V, Dell'Orco D, Manara E, Baglivo M, Fiorentino A, Maltese P E, Pinelli M, Herbst K L, Dautaj A, Bertelli M. Aldo-Keto Reductase 1C1 (AKR1C1) as the First Mutated Gene in a Family with Nonsyndromic Primary Lipedema. Int J Mol Sci. 2020 Aug. 29; 21(17):6264. doi: 10.3390/ijms21176264. PMID: 32872468; PMCID: PMC7503355.
Miller C W, Casimir D A, Ntambi J M. The mechanism of inhibition of 3T3-L1 preadipocyte differentiation by prostaglandin F2alpha. Endocrinology. 1996 December; 137(12):5641-50. doi: 10.1210/endo.137.12.8940395. PMID: 8940395.
Milne G L, Dai Q, Roberts L J 2nd. The isoprostanes—25 years later. Biochim Biophys Acta. 2015 April; 1851(4):433-45. doi: 10.1016/j.bbalip.2014.10.007. Epub 2014 Oct. 30. PMID: 25449649; PMCID: PMC5404383.
Pallai R, Simpkins H, Chen J, Parekh H K. The CCAAT box binding transcription factor, nuclear factor-Y (NF-Y) regulates transcription of human aldo-keto reductase 1C1 (AKR1C1) gene. Gene. 2010 Jul. 1; 459(1-2):11-23. doi: 10.1016/j.gene.2010.03.006. Epub 2010 Mar. 23. PMID: 20338228; PMCID: PMC2874818.
Penning T M, Wangtrakuldee P, Auchus R J. Structural and Functional Biology of Aldo-Keto Reductase Steroid-Transforming Enzymes. Endocr Rev. 2019 Apr. 1; 40(2):447-475. doi: 10.1210/er.2018-00089. PMID: 30137266; PMCID: PMC6405412.
Quinkler M, Bujalska I J, Tomlinson J W, Smith D M, Stewart P M. Depot-specific prostaglandin synthesis in human adipose tissue: a novel possible mechanism of adipogenesis. Gene. 2006 Oct. 1; 380(2):137-43. doi: 10.1016/j.gene.2006.05.026. Epub 2006 Jun. 10. PMID: 16842938.
Rizner T L, Lin H K, Peehl D M, Steckelbroeck S, Bauman D R, Penning T M. Human type 3 3alpha-hydroxysteroid dehydrogenase (aldo-keto reductase 1C2) and androgen metabolism in prostate cells. Endocrinology. 2003 July; 144(7):2922-32. doi: 10.1210/en.2002-0032. PMID: 12810547.
Rizner T L, Smuc T, Rupreht R, Sinkovec J, Penning T M. AKR1C1 and AKR1C3 may determine progesterone and estrogen ratios in endometrial cancer. Mol Cell Endocrinol. 2006 Mar. 27; 248(1-2):126-35. doi: 10.1016/j.mce.2005.10.009. Epub 2005 Dec. 9. PMID: 16338060.
Strait B J, Dewey T G. The Shannon information entropy of protein sequences. Biophys J. 1996 July; 71(1):148-55. doi: 10.1016/50006-3495(96)79210-X. PMID: 8804598; PMCID: PMC1233466.
Taketani Y, Yamagishi R, Fujishiro T, Igarashi M, Sakata R, Aihara M. Activation of the prostanoid F P receptor inhibits adipogenesis leading to deepening of the upper eyelid sulcus in prostaglandin-associated periorbitopathy. Invest Ophthalmol Vis Sci. 2014 Mar. 4; 55(3):1269-76. doi: 10.1167/iovs.13-12589. PMID: 24508785.
Torre Y S, Wadeea R, Rosas V, Herbst K L. Lipedema: friend and foe. Horm Mol Biol Clin Investig. 2018 Mar. 9; 33(1):/j/hmbci.2018.33.issue-1/hmbci-2017-0076/hmbci-2017-0076.xml. doi: 10.1515/hmbci-2017-0076. PMID: 29522416; PMCID: PMC5935449.
Volat F E, Pointud J C, Pastel E, Morio B, Sion B, Hamard G, Guichardant M, Colas R, Lefrançois-Martinez A M, Martinez A. Depressed levels of prostaglandin F2α in mice lacking Akr1b7 increase basal adiposity and predispose to diet-induced obesity. Diabetes. 2012 November; 61(11):2796-806. doi: 10.2337/db11-1297. Epub 2012 Jul. 30. PMID: 22851578; PMCID: PMC3478517.
Zeng C, Zhu D, You J, Dong X, Yang B, Zhu H, He Q. Liquiritin, as a Natural Inhibitor of AKR1C1, Could Interfere With the Progesterone Metabolism. Front Physiol. 2019 Jul. 3; 10:833. doi: 10.3389/fphys.2019.00833. PMID: 31333491; PMCID: PMC6616128.
Zhang B, Zhu D W, Hu X J, Zhou M, Shang P, Lin S X. Human 3-alpha hydroxysteroid dehydrogenase type 3 (3α-HSD3): the V54L mutation restricting the steroid alternative binding and enhancing the 20α-HSD activity. J Steroid Biochem Mol Biol. 2014 May; 141:135-43. doi: 10.1016/j.jsbmb.2014.01.003. Epub 2014 Jan. 13. PMID: 24434280.

1) A method of treating and/or preventing of human lipedema in a subject, the method comprising administering or applying to a subject in need thereof a therapeutically effective amount of a compound of natural or synthetic origin suitable for modulating the activity of AKR1C1 or of prostaglandins, wherein the compound is an inhibitor of AKR1C1 or modulates the catalytic activity of the AKR1C1 enzyme, and comprises competitive inhibitors with a core structure of flavones.

2) The method according to claim 1, wherein the step of administering or applying to a subject in need thereof a therapeutically effective amount of a compound of natural or synthetic origin is preceded by a detecting step for the diagnosis of lipedema wherein the detecting step identifies the c.160T>G (p.Leu54Val) variant in the sequence of AKR1C1 gene that confirmed the tested person is affected by lipedema.

3) A composition for the treatment of human lipedema in the form of a cream or ointment, comprising an inhibitor of AKR1C1 or a compound that modulates the catalytic activity of the AKR1C1 enzyme or of prostaglandins, wherein the inhibitor is a competitive inhibitor with a core structure of flavones.

4) The method of treating and/or preventing of human lipedema in a subject according to claim 1, wherein the competitive inhibitor with a core structure of flavone is a flavanone.

5) The method of treating and/or preventing of human lipedema in a subject according to claim 1, wherein the therapeutically effective amount of the competitive inhibitor with a core structure of flavone is in the form of a cream or an ointment.

6) The method of treating and/or preventing of human lipedema in a subject according to claim 5, wherein the competitive inhibitor with a core structure of flavone is a flavanone.

7) The composition for the treatment of human lipedema according to claim 3, wherein the competitive inhibitor with a core structure of flavone is a flavanone.


SEQUENCE LISTING (6 sequences)

SEQ ID NO: 1 - DNA, 19869 bp, Homo sapiens
  source 1..19869: mol_type = genomic DNA
aaacatttgccagccaggctagtgacagaaatggattcgaaatatcagtgtgtgaagctgaatgatggtcacttcatgcctgtcctgggatttggcacctatgcgcctgcagaggtaacaataatatttttagtgttgagagttcaaaggagctagagtaagtggaagctgaccaggttgtcaggcttgtgttcctatgttactctgcatgactcccctttaaacgtcagtctttgttctgcaatgccatcttgtcacagggtcatctactgcttatttgtggcactgttttgtcttctgtttatgtttatttcacagcttgtcagagtctatacaactcagcagaaagaacacggcttgcctgctccctctcttgaagattgattgcaatgggagtggtttctctgtttctttgtatagtcgaacagatatttacttcttccaagaagatacaattcagaggaatttttatggcaaaagttagtggagacggggtggatgaaatctgttgggagagtatcactgttctgatgggcggccttgagcaccacactgtaatactagacctggcctaaagagaatttctcatgtactattctcacctctgggaaaattacctgaaacgataaaatatcctcattttaagaaaaaaaaaatactagaacctaacaaggaaaatttaaattctttttctttgttaatatgggtcaaatttgtatcaaaatattgtaacagaaaaatttcttgtttctctgacaggaaaattaatggaatagattttcttttctagacttagctatttctacactggaagaaaacttgtatctcaagactgtcttgggaatacaatgtgtgtgaatagggactagttacataaaatttgggtttcacatttctgatctatacaatataaggatttacaaagggacattttgaagaccattgacaggagtggtttactttgtaatataaattgacatggagaggaagtaaagatgaggataccactaaagctccagctttggtaagcctttgagtactcatacagcaaagaaatgatcaaggatggtatgaggagagggctgtgagacccagtatctagttccttgtaattctatgggaaaaagaaagcctctgaaatactttccagataggaaaacagtttgctttgtttctgtttttgtcatttaaaagatatactttcaaatctgtatccaaatttgttacgtaagtcaataattgtaaatttcactgcaatcatatgtttcctaaaagagttgccttgaacttatggaactgaaccccattttgctaatcacttactccttgcagtctatttcttatacaaatcaatatccttagagtatacatttttacacctactatttagttctgtcatgccgacagaatggctaatggctgtgtagaaaagacttgacataatagactgctatccatagacaggcctgtttcccatataaacctgctggtatctgataaggtggatttctcactatttcctaactgataagaatggtggctgtaagtaactgcttgtgcaaactgcggagtttctgtgcacctgctttccttttggagacttttggaagttttgtgcctgcgaagcagagtgagtcaatgtaaccagtcccagacaaaatcttagtaatggagtatgtagagagcatctcttgtagacaacagttcacatacattgacaaaatatgattccgggtaaagtcagtgcattctgtgcaatatggtgggagaggaatcacaggagcttacactgtattccttcatatttccttccatgtggctatttcctcgtgttaatttctttttttgtttgtttggtttttgttttgtttttctgagatggagtctcgctttgtcgtccaggctggagtgcagtggtgcgacctcagctcactgcaagctccgcctcctgggttcacgtcattctcctgccccagcctccccagtagctgggactacaggcgcctgccaccatgcccggctaattttttctatttttagtagagatggagttgttaaccaggatggccttgatctcctgatctcatgatccgcccagctcggcctcccaaagtgctgggattacaggtgtgagccgccacgcctggccattccttgtgttaattttaccatactaaaaataattaagattatatctataactataggttgcatcctgttaatttttccaaaaaatgactgaacctgaaattgttttggggacacttccaaactagtggtcaagcagtgatttttctgggactgcagaagttcctgctgtgcccaacctttattactaactgggaaagacccagggagactgggatgggctcatgattctacatacagaactcatccaagaaaggaggaaaagctgatttttgtgaacgtcgctacttgtgcctgaactaactctcaggcacattagtcagaaaatactacctatggttactcccccaggttcctaaaagtaaagctttagaggccaccaaattggcaattgaagctggcttccgccatattgattctgctcatttatacaataatgaggagcaggttggactggccatccgaagcaagattgcagatggcagtgtgaagagagaagacatattctacacttcaaaggtactgtgcctatgatgagcttgtgtgcacatgtatttattgtgattgtgtggaggtggcagttctatgactggatccatagtatagggtgaattgtgcttatttattacgatttattcacacttactcatgtattaaaactaatatcaaaggcaggaagtgaagatggctttctcatctttgcagtgttccaattcatgccttcaaagtgcctttactttttgagctcagcacagatcaatatggtttaacatagactatagaatcagaaaaaatcctaatcatattatattatttcttacctcctgggtgagtttcttacacttgttatgattctcagtttccattattatgttgatcatagagagagaaagcaatataagcaagctctgtgagtattaaagaataacgcctacattatctcaaattgtgtccagcccaagttgacatattaaaaactctgtttgcttcctcagtttctaaaccacaaagatacttaatcttgcccattgggctgaatagatgaaataattacactactaacatgagttgtaaaacttaccaaagataattcagataatgggtatgcttatgatttgataacaacttttaccttttgagtaatttcactctttcattcaatatatgtactaactatatatccaacatattataaagttcatcatgatatggcagaacaaaaggcatcacaagaagagagagaataattttgcctgtggtcattagttttgaagtagtagaaaatgtctaaatattaggtggagcaaactagtaaaattggctcaagttttcattacacaacttcctttctctaacctctgcagctttggtgcaattcccatcgaccagagttggtccgaccagccttggaaaggtcactgaaaaatcttcaattggattatgttgacctctaccttattcattttccagtgtctgtaaaggtaggcagcttgtgtgatcaaattaatttcacttttgttctcagcataaatattgtttttatggatatttgaactaagcattttcttaggaggacatagggattataacatagaagaagaatcctaaatctaactcctaattcctttctatgggatacattttgaatccatacttccgtgattgcatgtctataagaaaagaaagtgcagaactctcaaagcctctgcctcaaaaacttgaggaaatgatagtcatctccttgaaggcacaaggtcttagttatgattcctgatttcacctcttgggatgttcacagacacagagtttcatgaagctgtggtgtccagaaaacctgctgcacatagggtgcacaatgagtttccatcttcttgcctctttttaaggggcgagaactcagtccgggagtgtcttaaactacagaccttcatgggaaaccttgttgcttctgcttcctctcttttcacactggaggttttatttttgcttagccatgaattcttgggtcattcataacttttgtcttaaggtactgaaaactagtcaggctagttaatgcaaaagggtatattagatatgataatgggaaatcaaagccagggctacattaagaatttgataaaaactctaaaaatatatcctttctccacccttatctctgctttattacaaaaggctttttaagtatttgttccaacctttttcgttggtggcatttatggctttggagcactgtcaggcccatgttcattaccgtgagctcctgtgcatctcctaatttccaaactagccttgaaaacacctccattgaccatgattggttcatggtcctgtgcatggaacatcatatgttcagggagataaagaactctgatagtggcacctgggtaaaaagtacaatccattatatctggatatcaagatcttttgcagttgaagagaggtattgccacagagaaaattataggagcagaagaaagtcaatgaaagtcaatgatgacactccattaggaaccagaaagaggtatttatttatacatgtaatagttgtaagagattagagggagcctgtcacctgaagacattccttataccttcatatgtagtacactctgtacataccactctctggagcactgcatcacctacctcatggaggattagtgtccttaaatgtacctcagagcatggctatgtgtggagaaattgacttgtgacatcactaaactgactgcttctacttcagccaggtgaggaagtgatcccaaaagatgaaaatggaaaaatactatttgacacagtggatctctgtgccacatgggaggtgagtgtttggaggtgagagaacggataagaaagatgaggtaggatacatctgtttcctatcttcttagtacaagtatggaaaatgcaccattggatcaaaaacttaggaactttacaaaggtagtttttgtgagcagtgaggaaaaagaaatgacaggcatacaaaagagagaagtggaggagaagaattgggggtaaggaggactgggatttatttccttgccttttcactaatctctccagtttcccaaggaggaaacagaaatacttactcttgcaatcactatcttcttccccaattttctgttttattttttcccttttaagaaccacggctagctagttttattgtcatatctagacagttgttactttcacagttctgtgtcacatttatcttgatcttccacacctcacaattcctttttcccaggccgtggagaagtgtaaagatgcaggattggccaagtccatcggggtgtccaacttcaaccgcaggcagctggagatgatcctcaacaagccagggctcaagtacaagcctgtctgcaaccaggtgagcaccctcagcctcctctcctttctgttcttcatgcccctctttctgtcctattgccaagtatccattcatttagtcccacttatctttgtaaaagagaagattctagagagcaaagactctgtctcgaagggcatagagggatcttacttgtacccggcttagaaaagtcttagaaaaggtgttgtaactttgatgctgaattgtgtgttatatttatgggaggtcaattcaatcaaggaggccaaggattgtcaattagactcagggaaaggtggacttacccctaagctatgaaagatgaccagagaatggatgggtgaaggtgatttggagggaactgtgcatagatatgaagccgtgaagagatggaatgaacagtacataaggagagagtgagagcaagagaagagaaatactagggattcacatgggcttcggatgtctcagtgcctcttggtgtctctgctttcacacttcttgaccaagggcatagatacactaaaaattatctgcagacaatgaaggtcatccatggtagaaagagaaaataacctgtttatgggagatatctgttcacagtggatgaaatccacactgaggaaggttcagcagaacatggagctgacctatgactgcccccgctcctagttggctgctcttctttggagtctgtcatgcaatcagctgcttaaacacatctattctctatgtgttccttttgtagcctagagataattccatcttttccttgagtcctgactggtgattccaggcctccttgatcaaattgactgtccccaaatgttacacacatttcagcattaaagttactacacttttcccagtaaattacatttttttgttttattttatgttttaaaacttagagtcaatcagtgaagatctacagtggaaatacatatgtataaaattagtgaatttggtataatgatacggtgctgtgataatttaataaaagcattgagaaattaatgtggttttaatactagtagttccccaattttttaatatggccattttcacactttccaaaaatttgataaaaataatactggagacccatgttaccatttatattcaaaattactaacattttgcaatgttatatggagtatatctgcataaatacatataatgtttctagaacctcttcaaggtaaattgtaaagatgacaattgacccctagagacctctcaaagcatgtcacaggaatgaagatattctcccagttccagtgtcattactgtatctaagaacatcatctatagtgccctcatgtcatttggtatccagtccatatttgaatttttccttgcttaatcaaaatatcttttgtagctcttctttttttaaccagaattcaattgtttttatattgttgtattttgatatcagttaacttgtttttcccagttgttgaaggtttctgtaaattagaagttagatgagagtgttttattacattgacttaaacctttggggcaagaaaatatttttatcatgctgtgtttttcaaattgcaccacactagactgcatacaatcacagaatgccccactagtggtgaaggtagttttaaaaacttggttaagctccaacaatgatagactggattaagaaaatgtggcacatatacaccatggaatactatgcagccataaaaaatgatgagttcatatcctttgtagggacatggatgaaactggaaatcatcattgtcagtaaactatcgcaagaacaaaaaaccaaacaccgcatattctcactcataggtgggaattgaacaatgagatcacatggatacaggaaggggaatatcacactctggggactgttgtggggtggggggaggggggagggatagcactgggagatatacctaatgctagatgacgagttagtaggtgcagcgtaccaacatggcacatgtatacatatgtaactaacctgcacaatgtgcacatgtaccctcgaacttaaagtataataaaaaaaaaaaaacttggttaagctaacagtggtcagatctgttgtttttaactcatgttttctctttaacaattactagtaatctgctgggggataattctcaacagaagaagtcatattcccatgacaaattttcacatatgattgagtatacatcagtgatttgaacttttttctactgcatttctgttttcttctttgttatttcttatttgttatttccttggggtttttacataatcatttacaattttaaccacttgtttaactattcttacctgacatttaattaaaaatgatttctcaccaagtaaaaataataaactactattcctctaagtgagggtaaaggtgaacttcttccttcaattagcaatgtactgtgaacgtaattactgtaatgaccaccatcagagtagacaaagattgttgttctttttctcattgtcgcactaattactacagactcacggattcttgtgttatgatttttaatttattttactcattttttaatgctcacatctactcagcatatatatattatatatatatatatataaaatatatatgaaagaagaatgtgatcaaagactcatgagggcaagtgtaaaagacaaaagcacttgtttcatagggaacccactggtgacatttgagatatcaacatacaaatccatcatacaaaaaggaaaagtaatgaaagactgtgaaaattatcagtatttatgtgtgtatgtatattttctagttcatctatatagatatatatcatagatggcatatatgatatatatattatatgtcagatcaattagaaaacatacatatacatgtatgcatttttggtattaattaggaaactagttcttttgtctttcagacctgccctcatgagtctttgaccaaattcttgatttctggcacgatttgatgcccaaggctcaaagaatcttacctcatcccagctctgaaattggccgtgtctcgttctagtggtcggagttattgagaaaccaggatagggttcatggagttgcttgtgctcagaaatagcatttctattttcttaggagagaggggcataaaatttatttttatgaaaaagatcatttttgacaataatattctcagttcaaatttaatgctctacattattcagcttcttttactttggtgattttaatattagtgtaacttttggattatctgatgcttttccatcttgctcgtctgcaggtggaatgtcatccttacttcaaccagagaaaactgctggatttctgcaagtcaaaagacattgttctggttgcctatagtgctctgggatcccaccgagaagaaccatggtaataagagatacaggaagtttacctaaaacaccggtttgataaaaaaaatttaatcagatcatgctgtttcctggagttcactcacagctgacttggggtgagggaagaatttgcatttctgacgagatcccaggtgatgttgaggctgctgttcgggggcctcgcttgagaagctccggtgcagagtggacgccttagtctgtttagggagccgcctaacaaactgtatccccagcctcagggcctcagcctttctgcctttccttccagggtggacccgaactccccggtgctcttggaggacccagtcctttgtgccttggcaaaaaagcacaagcgaaccccagccctgattgccctgcgctaccagctacagcgtggggttgtggtcctggccaagagctacaatgagcagcgcatcagacagaacgtgcaggtgaggagcggggctgtgggcctcaggtctcctgcacagtgtccttcacacgtgtgcttcttgtaaggctctcaggacagccttgggccagctccatttccctgtatttcctatgcatgaactctttgtgtacgtcataagggtttcttctactctagcacaggagaggcaacagaggtggagagaaataggatgggatcaaaactacagatttgggttagcgttaagtcagtgatatccatatccctctgctggggccatgtccttttctttattttttataatgaagcattgattagatgttctttagtctccaactcatggcggatttttcttcttgtatgtttatgtagtataaattccttatatttattctcttagaattttatctttgcgaagtgcttgtttaactctgcctttttttttttggcgaggtggttgtgtgtctttatttttatgtgtaggagaccatatttataccaataccttaggatataagacaaacccacttaatatagaggcttaatgcaaaagcatttattgagctcaaggttccatggttagaaaattagcctgggctcagcctaaactcagtccattccttctgttcctggctgggctctcatgtgtgttcagggcagtgaggcagctctgcttcaggatgtccctggggcatctgtgtgagcgcccccctctcccatgcagttctttatcctccagccatccagcccagggttcacctcagcacagggcatgaccccaaggcagtgagtggacatgtgcaaagccttttcaggtctaggctcagagctgccaaccattgcttctgggaaattctatcacctaatgcaagttacaggacaggtcagactcaaggggtggggaaaaagacaccacctcttgttggaaggagctggaaattcatataaccaagcaagtggatacagggacatgtgattgttagagggcatgttcctaattaatctatcatttatacgatatgtgtgaaataacatattaatagttatatgtatgaatatgaatatatataaattgatatactatttcatatattcttaagaatacatccaagtctctaaagaattaatatccagaatacatgcatatatatgttttctaattagttatatacagatatatgaaatatcatatatgatatatattataaaacctatgtcacatatatgacatatatcatgtatatgtatatgtgtcactatttgctaaaacattaagtaaaatcagtgtagaaatccataggatactctgaaataatgatacagagaatgagaaagaaagtaaaaaatgacaatgaaacaattgatgaatctaggtaatggcatttactatattattttaaaaatatttttagacattagaaaacccacaaaactaaaaggaaatattatgaaataactgtggaaattatcagtatttatacatatgtgtatgttctctaattcatctagtatatatagatatatgacatatgtgttatataatatatatgttagattaactagaaaccatacatatatatgtatttattctggatattaattctttagagacttaaatgtattcttgaaataacatctcccaaattttacctgtgttgtcatattctctgtggttatgttttcacaaacaaaagtttctatccttcatgtagactaatttttcatgcctctttcttgtggttggtgagtgttgtgtcttatcctatattgggaatatcacaatatattactgtgttatcttacaaaattgttaatattctttccttataaatttggttaattctaatgggattgactttttaaatactgtatgagataaggatccaaatatgcatatctaatttttgaatttttagcaaaatttcccaccaacatttattgaaaagttcatatttgctcacataacttgatttttttacctgtaacatatatgaaatgtccatgagcaattatctggcctctcttccatcttcagttaacttatttacttttctgtactatgatgttaaaaagtaagtttcacttccagtatggtacaaatatttttctaacttcttcatttatttaataatatcttagctattcttgactaatgtgttatttaaaactttgaatcatcttattaacttccacaagcaaataaatgtttagtctcttgttgacattgtaataagcctccagatcaatgtggagagaactaaatgtttgacaatattgagatttccaatgcatgatcataatatgaatttcccacttattaatagccttttaataaatatataattaatttcataactttctctgttactgatgattttaatagtttttgataacttttcatttttgttttatggattttctaatatatagaaatattaaaagaataatgcaacaaatcttcttacctacattcatcaagtgttacattgctgtcattttttactctctttctctcattttctgtattactccttcagtttccctatggatttatacacttatttgattgaatgtattagaaagagtgacatctttttgatatttagagtctcccaaatttacatagtgagagtacccacaccttttgaatgtgaaatcgttagtatttagacaattattggcttgagtgatgttgaaaactaatttttcctattatacctgttcattggagagatagatattcaatgttattcatcaaacctactaaattatgctagacattacaaaatttgcctctagattcttgtgggtcttctaggtacatgaccctatcatgtgggcacaaagtaagctctgtttcttcttcagcttcctctgagatattctcttcatctgcagtgttgtgcagtttcaaaatattttatctgggaatggatttctgcttatttttgactggttttcattgaccacttgggtgtttagagaggtctttcataaattttggaaacgtatcaatcttttaagtattgtctctgcaccctactgtctgatgaacttggttgttcccaattggcaatgatgtaatctaaaaataattaaagttttctattactttgataggtgtttgaattccagttgacttcagaggagatgaaagccatagatggcctaaacagaaatgtgcgatatttgacccttgatatgtaagtaattttggagatggttgtcctaatttattttcagaggaggaacgtaggatgggtgttgagagtgacctccacaccaggggcacagaggccaacgtgagacagaggtgagacaggagctttctggaagtctcctcctggattcactccagagctctgttctctggcagggagagtggcctggggtcagcatgggtcaacctgtgcctctgctctcgtgactccaaggaactttccagagcagccaagatcattgctgaatctgcaccttccatgtaggcctgctgtttgtgctactgtctagtgtacacactgtggatattgcccatgtggtcgcattagatgtttccaaatctgtgcttctatcttgttcttcccaacctgctcaatgtcttaccagatcaaaggcagttccgtcaatcttgtgggccaggttccaattcccacctctttcacatggaattgcttgctggatcctgtcgatttagcatttttaatcatttcaaacctttttcgtgcctgtctttgcatgtttgcccatgagccaaacactgcatttgcctccagaaagtctgtcttagtgaagcgaataggagcacttggccttggtgttctgcaatatggagatctcagtgcagagaatgaagaaatgttaaaatcaaatgcagagtgtagatgcaaaagcaccattgttggagataatttataaccaataacagaagtttactattcctctgtgttctaaggttctaggattcatagaaggtcattcatataatgagggaaaaaagaagttatgtaactgctggcagtagggatttcaacaagtaatagagatgatgagataatgtgtgaaaaaaacaagcacaatggtacatcataagtacccaaaatttggtggatattattagtagtattttattcatttagaggaaaatacaaaatgaaaacggagatgataaaaagaaaaggaaaagtggcaagcattagaaagttttaaaaccaattaatgtttttggggatcatgaaatcaatcactagtgttattttgtagaactcaaatatggtctggagtaaaccacttgcgagcttccaagtgtttgctcttggtgtaatcaaaaaggataggctcaattccctaattaataagttgtaattgtacatgtgagcagtcatttatctgtgtctttacattttgtgttatttctgcctataaaatggtttatacattgtagctctttggatattggaccctatatcaaaaacaacaatttacatttctgaatctaaccaactatgttgcatatgataggcaggtagtaatatgtaaagtaattaagtataattgctaccagtataatcatcacactcaaaattgtttattagtgatgagcatatgtcttgcttattcctcttcaatgtaaggcagtatagttccatttttactgagaatttgaaatagaaaaggtcagtatatctctttgctctgttgacagatgtagagaaatatgtttgaacatttcactttgtgtgttttatgtgttaagttccaggcaggggaatagaattaaattattctttattttgaaaatacagatatgataaagtcacattcattaaacaaagaaacacagattctagagcagtcagaaaatgaacttcttaacatctacactagcggcagcttcctagaaatcactgcactacccgctagtaacggagtcattgccattcagagtgtgcatttttttttctctttccagttttgctggcccccctaattatccattttctgatgaatattaacatggagggcattgcatgaggtctgccagaaggccctgcgtgtggatggtgacacagaggatggctctatgctggtgactggacacatcgcctctggttaaatctctcctgcttggtgatttcagcaagctacagcaaagcccattggccagaaaggaaagacaataattttgttttttcattttgaaaaaattaaatgctctctcctaaagattcttcacctactttggtctccataacttctatgttttctttccttctgacacactagtgcccctaaattgtgatttgcctatacgtttagggccggggttggaagatgttaacaaccatttaagattcatttctgcagtgggagtgggtggagtttcaccctctgggaaaggggcaggtgacaggtatttatcagtcagtgcctctctagctcttgtaggaagaagcacacgcaggatggagtctagaggatgagcgatattgactagcaattcatgggctccctccagcagtgcgagggtcagagtttctggagccttgggaggaggcatccctgtgagggggggttagggagatgggagggcaccaggaaaagtgattagaagtcaggtatgggaaggctaaataggacagagtcgagtacatctctgcttggaaaaacatatcaacacccttttttttgaacattatatcttgctcataaaagaaaactttccacattgttttaacaaaccccacagctgagagtcaggcctgaatctttgatgtgtgcccattcacaacgttgaccctattggtttgtggtggggcagggcatcaaagacatcattgactaatcacattcccctgaatagctcatatttagaaaatattcttagattctaaaaatgtactattaatttgtgatattcagtcttttaaatattttatacattaaacaggcatagttacaaatataaaacaaaaatatcccaaagccattatgcatggcactcaagattaaaatgggaaataatacatctaataaatcaaatgttccaagacttcaaaggtcttttggaaacaggctatgtaaaacagcacactggtttcaaactttggtaaattttaagaacaactcttacaaaggcatttaattcttatacataattttcaggggacctaagttaatcagctaatcatgaagacatgattttcattttagaaaacacttttgaaaacttgggataatctcatgccttaatgatcaaagcattatgagaaggacagtggtttttaacctgggcatatgttctaacacatttactctccactattcgtactctggtagccatgttaaccccatcagagattccttctcaagccatgtctcagagctgagaggcatcccagcaagttttgcagctcacagttttttccgtaaattacttattctataaaattggagtaggccataaactttggagggccctagaccaattttttggattatttttcgtcttctatcattccgctgatcttagatattctctgcattaaatattaaatatcacttctaggctgaaaaatccccctaaaaatatttctagctcagatttttcctccaaattctgcaatagaagatcacaatgtgaactctgcatctccatgttaaagtctaatggacattcacacttagcatgtctcaaagaaatctcatgtaaaccatggccatcctgttctaccttaactttctgagtctatggaatgataatttcacatctcataaacttgactgatgtaagtgtcaagaaaagattgacattttgttaaaagttagtagtgaagtgtgtaacgcttaagcaaactttcatatttcaaatctctttagcaagtgtaactcttttttcaagatgtgaaataatcattaggtcagtcatttgtaaatagtacatctgctatggactttttccagttcttcaccatccatttttataaaactcttattgttaaaaaaaaagttactcagaatttcataaagccaaacacctgatttcaggaacacttgagatgtaagaaaattttatagggacctccaatcactaattttcctattttttctctcaaagaaatgctgaagggaggaattcaggttgaatgaaaggaaatagtaacttacagccatatagagttataaagacttcttgtaaatgtgaacatatggtaaaatataaaaacatgtatttttgaaattttggattctactcattattttacttcaatttaagatataaatgtatagaaataagtataattctaagctaatacgtatgcaatgtaggaagctgtaattactgaccaaaactatgtgaagtggagaaaacctggggaagtggatggttttagatgaaactgaagttaaattcatattgatttaaagtaaattgttataactttataaagtttttcatcatcaccacagcaatcacaaagagaataattatgaatatacgcaagaggaaatgagaagggaatccaaatgtcattaaaaaaaaatcacgccacctcacaaaatggtaacagtggatataaaggatagaaagctataaacctggtaggaaaacattgaaaatggcacaagtgactccttccctgtcaccaatgacagtcatgtgttgcataatgcctgggctatcttctgagaatggctttgttaggcaactgtgtcatggtggggacatgatagcattagagagaattctctcaaacactgtcatggccttatcaactatgcttctgtaatattctgaatccttcgctgcctttccaacagtgtttacaacggtttcctaagaaatgaaatccatttcaccaaaccacttttcttgctcatccataagaaacacttctcctttgttaaaattacatcatgagattgcaagcattcagtaacttgcaagctccaatttgaattctagttgtcttgctatttccaccacatctgcaattactttctccactgaaatcttgcattcctccaagtcatccaggagggttgaattcacttctttcaaactcctgttcaagtaaatgttttgccctcctgtcacgaatcatgaatgtttttaatggcatctagaatagtaaatctttttcagaagattttcaattttcttttcccaggttcttcagacaagtcaccatggtagctgtagacttatgaaatgtatttcctgaaccataaaacaagaacatcaaaattactctatgatccatgggaaacagaatgcatgttgtgttagcaggcatggaaagaacagtaatctctttcaacctctgtaagagagttcttggttgattaggtaccttgtcgataggcagttctaatttcaaagaactttttcttttgctgagcagtaagtcttaacagtgggcttaaaatattcagtaaaccattatttaaggagatgtggcttaaatatctaggctttcttgctttatttatagaacacttacagagtagatatagattaaatcttaaggaccctaggattgttagaatagtaaatgaacatcatatgttgtaatatagttagccagggaggggaaagatctctacaagaaataattttaaaaactactcaaagaaatcagagatatcacaaaatgacaaaacaagtacaggggcagcaaaaggtaacagatgtcaactatgagatctcatcaagaactaagaaaatggagaaaagtaccagatggcagatgggaggcagtgtgagcatgcctctcccacttggagattagtgtgtagagatttacattgtgaatttcttttcaagaaccaatgcagagacttaacagatatatgaaaaaatttcacatacactctgaagaagtctcaggcacaatcctactccatgaaataagaaaaaaccatcacatccccagattctgaggggagagagttcactccagagcatacatccccactggggatctggaaattcaggccacaagagaaggctttgatcctgtgcggacaagtgccttgagcagaatccacaggggagagctggaagtgcatggcagatatgagcacagaagctgggtgtccagccttgtgggcagaccgggagggatgtggcctgaaagccatggttattatctcagcgtgtaagctgatgatctgaggctggtcagagttctgtgcgcagactgcctggatgtaaacctggcactgttagcagagcactgcatgaatgaaaggaatgaaaatagccttgccaactgcataggtgctgggtgaggctcactgtggggggcgcacggcaagctattcaacattacgtacaggcatttgaggtcggggcatggaaaaatactgaggcagtgtgtgtatgttatttgtgctgagaatgaaactccttgaccttgaaaacaggacagggagtggtgtgtgtgctgtgataaggaatgctgaaaacggtctcctgagaatgcggtttgagtgcttttacaaggccacaggtgtctcacgacctgacctcataaagccatctagtggatgtttgtggcttaacaagccccttcaataagtacttggcggagggatgctggggggaccttcttagaagagctgcgccccacccccctccctggacagcccagctacaattgtctgagaactcactgcaagctgtaagcggcccactacttcccactcccttttttgaactcttctgtgcagcagaggcaattaaactctcctctggaacattaccccagcagcctgagaaccaccccttggcgctcacaggggctgcagcctgccttatccaaggacagttagagtgcagacccacctaaccctggacccactggtggtacccatccacccatcctggtagcttaacacaaagatacacttttaggagcttcatagcccctcttcccccattgcctgagaaaccactttccctgggcaacttagggcaagctcaaatcccactgctaccaccacagctggtgctcttttgcaagtgccacctcctagctggagaccaaccagcacagtccacacagcacctcaaggaagaataaatagaactgttcccaggaaggagaaaatgcctgcatgagctcagctgttaccactgcgtaccacaccctgaccagtcagaggtcttgagtcggtccgcatgacaagttcaccgctcgcataaccagcattcaagaaagccagcacactaagcctatctacagccagggagtctcagagtctacgtcactcccctgccacctcaatcagagctggtgctggtatccactgctgggagacttgtgctgaggtgaagcctgggctggatggctggaggatgaagaactgcacggaggagatggaggcacccagacagatgccccatggacatcctgaagcagagctgcctcactgccctgcacacacatgagagcgcagccaggaacagaaggactgggcaacattcaggcggagcccaggccaattttctaaacatggaatatgagttaaataaatggcttcgtattaaa

SEQ ID NO: 2 - DNA, 6513 bp, Homo sapiens
  source 1..6513: mol_type = genomic DNA
atggattcgaaatatcagtgtgtgaagctgaatgatggtcacttcatgcctgtcctgggatttggcacctatgcgcctgcagaggttcctaaaagtaaagctttagaggccaccaaattggcaattgaagctggcttccgccatattgattctgctcatttatacaataatgaggagcaggttggactggccatccgaagcaagattgcagatggcagtgtgaagagagaagacatattctacacttcaaagctttggtgcaattcccatcgaccagagttggtccgaccagccttggaaaggtcactgaaaaatcttcaattggattatgttgacctctaccttattcattttccagtgtctgtaaagccaggtgaggaagtgatcccaaaagatgaaaatggaaaaatactatttgacacagtggatctctgtgccacatgggaggccgtggagaagtgtaaagatgcaggattggccaagtccatcggggtgtccaacttcaaccgcaggcagctggagatgatcctcaacaagccagggctcaagtacaagcctgtctgcaaccaggtggaatgtcatccttacttcaaccagagaaaactgctggatttctgcaagtcaaaagacattgttctggttgcctatagtgctctgggatcccaccgagaagaaccatgggtggacccgaactccccggtgctcttggaggacccagtcctttgtgccttggcaaaaaagcacaagcgaaccccagccctgattgccctgcgctaccagctacagcgtggggttgtggtcctggccaagagctacaatgagcagcgcatcagacagaacgtgcaggtgtttgaattccagttgacttcagaggagatgaaagccatagatggcctaaacagaaatgtgcgatatttgacccttgatatttttgctggcccccctaattatccattttctgatgaatattaacatggagggcattgcatgaggtctgccagaaggccctgcgtgtggatggtgacacagaggatggctctatgctggtgactggacacatcgcctctggttaaatctctcctgcttggtgatttcagcaagctacagcaaagcccattggccagaaaggaaagacaataattttgttttttcattttgaaaaaattaaatgctctctcctaaagattcttcacctactttggtctccataacttctatgttttctttccttctgacacactagtgcccctaaattgtgatttgcctatacgtttagggccggggttggaagatgttaacaaccatttaagattcatttctgcagtgggagtgggtggagtttcaccctctgggaaaggggcaggtgacaggtatttatcagtcagtgcctctctagctcttgtaggaagaagcacacgcaggatggagtctagaggatgagcgatattgactagcaattcatgggctccctccagcagtgcgagggtcagagtttctggagccttgggaggaggcatccctgtgagggggggttagggagatgggagggcaccaggaaaagtgattagaagtcaggtatgggaaggctaaataggacagagtcgagtacatctctgcttggaaaaacatatcaacacccttttttttgaacattatatcttgctcataaaagaaaactttccacattgttttaacaaaccccacagctgagagtcaggcctgaatctttgatgtgtgcccattcacaacgttgaccctattggtttgtggtggggcagggcatcaaagacatcattgactaatcacattcccctgaatagctcatatttagaaaatattcttagattctaaaaatgtactattaatttgtgatattcagtcttttaaatattttatacattaaacaggcatagttacaaatataaaacaaaaatatcccaaagccattatgcatggcactcaagattaaaatgggaaataatacatctaataaatcaaatgttccaagacttcaaaggtcttttggaaacaggctatgtaaaacagcacactggtttcaaactttggtaaattttaagaacaactcttacaaaggcatttaattcttatacataattttcaggggacctaagttaatcagctaatcatgaagacatgattttcattttagaaaacacttttgaaaacttgggataatctcatgccttaatgatcaaagcattatgagaaggacagtggtttttaacctgggcatatgttctaacacatttactctccactattcgtactctggtagccatgttaaccccatcagagattccttctcaagccatgtctcagagctgagaggcatcccagcaagttttgcagctcacagttttttccgtaaattacttattctataaaattggagtaggccataaactttggagggccctagaccaattttttggattatttttcgtcttctatcattccgctgatcttagatattctctgcattaaatattaaatatcacttctaggctgaaaaatccccctaaaaatatttctagctcagatttttcctccaaattctgcaatagaagatcacaatgtgaactctgcatctccatgttaaagtctaatggacattcacacttagcatgtctcaaagaaatctcatgtaaaccatggccatcctgttctaccttaactttctgagtctatggaatgataatttcacatctcataaacttgactgatgtaagtgtcaagaaaagattgacattttgttaaaagttagtagtgaagtgtgtaacgcttaagcaaactttcatatttcaaatctctttagcaagtgtaactcttttttcaagatgtgaaataatcattaggtcagtcatttgtaaatagtacatctgctatggactttttccagttcttcaccatccatttttataaaactcttattgttaaaaaaaaagttactcagaatttcataaagccaaacacctgatttcaggaacacttgagatgtaagaaaattttatagggacctccaatcactaattttcctattttttctctcaaagaaatgctgaagggaggaattcaggttgaatgaaaggaaatagtaacttacagccatatagagttataaagacttcttgtaaatgtgaacatatggtaaaatataaaaacatgtatttttgaaattttggattctactcattattttacttcaatttaagatataaatgtatagaaataagtataattctaagctaatacgtatgcaatgtaggaagctgtaattactgaccaaaactatgtgaagtggagaaaacctggggaagtggatggttttagatgaaactgaagttaaattcatattgatttaaagtaaattgttataactttataaagtttttcatcatcaccacagcaatcacaaagagaataattatgaatatacgcaagaggaaatgagaagggaatccaaatgtcattaaaaaaaaatcacgccacctcacaaaatggtaacagtggatataaaggatagaaagctataaacctggtaggaaaacattgaaaatggcacaagtgactccttccctgtcaccaatgacagtcatgtgttgcataatgcctgggctatcttctgagaatggctttgttaggcaactgtgtcatggtggggacatgatagcattagagagaattctctcaaacactgtcatggccttatcaactatgcttctgtaatattctgaatccttcgctgcctttccaacagtgtttacaacggtttcctaagaaatgaaatccatttcaccaaaccacttttcttgctcatccataagaaacacttctcctttgttaaaattacatcatgagattgcaagcattcagtaacttgcaagctccaatttgaattctagttgtcttgctatttccaccacatctgcaattactttctccactgaaatcttgcattcctccaagtcatccaggagggttgaattcacttctttcaaactcctgttcaagtaaatgttttgccctcctgtcacgaatcatgaatgtttttaatggcatctagaatagtaaatctttttcagaagattttcaattttcttttcccaggttcttcagacaagtcaccatggtagctgtagacttatgaaatgtatttcctgaaccataaaacaagaacatcaaaattactctatgatccatgggaaacagaatgcatgttgtgttagcaggcatggaaagaacagtaatctctttcaacctctgtaagagagttcttggttgattaggtaccttgtcgataggcagttctaatttcaaagaactttttcttttgctgagcagtaagtcttaacagtgggcttaaaatattcagtaaaccattatttaaggagatgtggcttaaatatctaggctttcttgctttatttatagaacacttacagagtagatatagattaaatcttaaggaccctaggattgttagaatagtaaatgaacatcatatgttgtaatatagttagccagggaggggaaagatctctacaagaaataattttaaaaactactcaaagaaatcagagatatcacaaaatgacaaaacaagtacaggggcagcaaaaggtaacagatgtcaactatgagatctcatcaagaactaagaaaatggagaaaagtaccagatggcagatgggaggcagtgtgagcatgcctctcccacttggagattagtgtgtagagatttacattgtgaatttcttttcaagaaccaatgcagagacttaacagatatatgaaaaaatttcacatacactctgaagaagtctcaggcacaatcctactccatgaaataagaaaaaaccatcacatccccagattctgaggggagagagttcactccagagcatacatccccactggggatctggaaattcaggccacaagagaaggctttgatcctgtgcggacaagtgccttgagcagaatccacaggggagagctggaagtgcatggcagatatgagcacagaagctgggtgtccagccttgtgggcagaccgggagggatgtggcctgaaagccatggttattatctcagcgtgtaagctgatgatctgaggctggtcagagttctgtgcgcagactgcctggatgtaaacctggcactgttagcagagcactgcatgaatgaaaggaatgaaaatagccttgccaactgcataggtgctgggtgaggctcactgtggggggcgcacggcaagctattcaacattacgtacaggcatttgaggtcggggcatggaaaaatactgaggcagtgtgtgtatgttatttgtgctgagaatgaaactccttgaccttgaaaacaggacagggagtggtgtgtgtgctgtgataaggaatgctgaaaacggtctcctgagaatgcggtttgagtgcttttacaaggccacaggtgtctcacgacctgacctcataaagccatctagtggatgtttgtggcttaacaagccccttcaataagtacttggcggagggatgctggggggaccttcttagaagagctgcgccccacccccctccctggacagcccagctacaattgtctgagaactcactgcaagctgtaagcggcccactacttcccactcccttttttgaactcttctgtgcagcagaggcaattaaactctcctctggaacattaccccagcagcctgagaaccaccccttggcgctcacaggggctgcagcctgccttatccaaggacagttagagtgcagacccacctaaccctggacccactggtggtacccatccacccatcctggtagcttaacacaaagatacacttttaggagcttcatagcccctcttcccccattgcctgagaaaccactttccctgggcaacttagggcaagctcaaatcccactgctaccaccacagctggtgctcttttgcaagtgccacctcctagctggagaccaaccagcacagtccacacagcacctcaaggaagaataaatagaactgttcccaggaaggagaaaatgcctgcatgagctcagctgttaccactgcgtaccacaccctgaccagtcagaggtcttgagtcggtccgcatgacaagttcaccgctcgcataaccagcattcaagaaagccagcacactaagcctatctacagccagggagtctcagagtctacgtcactcccctgccacctcaatcagagctggtgctggtatccactgctgggagacttgtgctgaggtgaagcctgggctggatggctggaggatgaagaactgcacggaggagatggaggcacccagacagatgccccatggacatcctgaagcagagctgcctcactgccctgcacacacatgagagcgcagccaggaacagaaggactgggcaacattcaggcggagcccaggccaattttctaaacatggaatatgagttaaataaatggcttcgtattaaa

SEQ ID NO: 3 - protein, 323 aa, Homo sapiens
MDSKYQCVKLNDGHFMPVLGFGTYAPAEVPKSKALEATKLAIEAGFRHIDSAHLYNNEEQVGLAIRSKIADGSVKREDIFYTSKLWCNSHRPELVRPALERSLKNLQLDYVDLYLIHFPVSVKPGEEVIPKDENGKILFDTVDLCATWEAVEKCKDAGLAKSIGVSNFNRRQLEMILNKPGLKYKPVCNQVECHPYFNQRKLLDFCKSKDIVLVAYSALGSHREEPWVDPNSPVLLEDPVLCALAKKHKRTPALIALRYQLQRGVVVLAKSYNEQRIRQNVQVFEFQLTSEEMKAIDGLNRNVRYLTLDIFAGPPNYPFSDEY

SEQ ID NO: 4 - protein, 297 aa, Homo sapiens
MDSKYQCVKLNDGHFMPVLGFGTYAPAEVPKSKALEATKLAIEAGFRHIDSAHLYNNEEQVGLAIRSKIADGSVKREDIFYTSKLWCNSHRPELVRPALERSLKNLQLDYVDLYLIHFPVSVKAVEKCKDAGLAKSIGVSNFNRRQLEMILNKPGLKYKPVCNQVECHPYFNQRKLLDFCKSKDIVLVAYSALGSHREEPWVDPNSPVLLEDPVLCALAKKHKRTPALIALRYQLQRGVVVLAKSYNEQRIRQNVQVFEFQLTSEEMKAIDGLNRNVRYLTLDIFAGPPNYPFSDEY

SEQ ID NO: 5 - DNA, 19869 bp, Homo sapiens; identical to SEQ ID NO: 1
  source 1..19869: mol_type = genomic DNA

SEQ ID NO: 6 - protein, 323 aa, Homo sapiens
MDSKYQCVKLNDGHFMPVLGFGTYAPAEVPKSKALEATKLAIEAGFRHIDSAHLYNNEEQVGLAIRSKIADGSVKREDIFYTSKLWCNSHRPELVRPALERSLKNLQLDYVDLYLIHFPVSVKPGEEVIPKDENGKILFDTVDLCATWEAVEKCKDAGLAKSIGVSNFNRRQLEMILNKPGLKYKPVCNQVECHPYFNQRKLLDFCKSKDIVLVAYSALGSHREEPWVDPNSPVLLEDPVLCALAKKHKRTPALIALRYQLQRGVVVLAKSYNEQRIRQNVQVFEFQLTSEEMKAIDGLNRNVRYLTLDLFAGPPNYPFSDEY